(19) United States
(12) Patent Application Publication
(10) Pub. No.: US 2009/0018059 A1
(43) Pub. Date: Jan. 15, 2009

(54) Method for maintaining pluripotency of stem/progenitor cells

(75) Inventors: Paul Robson, Singapore (SG); David Rodda, Singapore (SG); Huck Hui Ng, Singapore (SG)

Correspondence Address: FOLEY & LARDNER LLP, P.O. BOX 80278, SAN DIEGO, CA, 92138-0278, US

(73) Assignee: Agency for Science, Technology and Research

(21) Appl. No.: 11/661,957

(22) PCT Filed: Sep. 2, 2005
(86) PCT No.: PCT/SG2005/000304

§ 371 Date: Apr. 21, 2008

Related U.S. Application Data
(60) Provisional application No. 60/607,479, filed on Sep. 3, 2004.

(51) Int. Cl. A61K 38/17; C12N 5/08; C12Q 1/02; A61P 35/00; C12Q 1/68; C40B 30/06

(52) U.S. Cl. 514/12; 435/366; 435/375; 435/29; 506/10; 435/6

(57) ABSTRACT

The present invention relates to a method for maintaining pluripotency and/or self-renewing characteristics of stem/progenitor cells. The invention also relates to a method for modulating gene expression in a cell. The methods include contacting at least two transcription factors, or a functional fragment thereof, with the promoter region of the nanog gene. One of the at least two transcription factors is selected from the POU- and homeo-domain-containing transcription factors. Another of the at least two transcription factors is selected from the HMG domain-containing transcription factors. The method further comprises allowing the at least two transcription factors to form a complex with a specific binding element within the nanog promoter. The complex thus formed regulates nanog gene expression by mediating transcriptional activation.

The present invention relates to a method for maintaining pluripotency and/or self-renewing characteristics of stem/progenitor cells. The invention also relates to a method for modulating gene expression in a cell. The methods include contacting at least two transcription factors, or a functional fragment thereof, with the promoter region of the nanog gene. One of the at least two transcription factors is selected from the POU- and homeo-domain-containing transcription factors. Another of the at least two transcription factors is selected from the HMG domain-containing transcription factors. The method further comprises allowing the at least two transcription factors to form a complex with a specific binding element within the nanog promoter. The complex thus formed regulates nanog gene expression by mediating transcriptional activation.

### BACKGROUND OF THE INVENTION

Stem cells have been shown to hold a key to regenerative medicine. This is due to the fact that they provide a source of cells that are able to replace corresponding tissue that has been damaged due to disease, infection, or congenital abnormalities. This is due to the fact that stem cells are undifferentiated cells that are able to differentiate into mature functional cells, such as heart, liver, brain cells etc., while retaining the ability to proliferate indefinitely. Gunther's disease, Hunter syndrome, and Hurler syndrome have for instance been treated by means of stem cells. Cancer patients with conditions such as leukemia and lymphoma have been treated with adult stem cells from bone marrow. The haematopoietic system of cancer patients, who suffered from cytopenias due to escalated doses of chemotherapy, could be restored by intravenous infusion of stem cells. Researchers in South Korea are reported to have successfully used adult stem cell from cord blood to enable a paralyzed woman, suffering from a spinal cord injury, to walk with the aid of a walker. Numerous further diseases are presently thought to be treatable by therapeutic transplantation of stem cells or cells derived therefrom, including Parkinson's disease, cardiac infarcts, and juvenile-onset diabetes mellitus.

A particularly viable tool in this respect are mammalian pluripotent stem cells, since such cells are able to differentiate into any organ, cell type or tissue type, at least potentially, into a complete organism. Such pluripotent stem cells are found in both preimplantation embryos and many adult tissues. Particularly prominent are embryonic stem cells (ESCs) that have been derived from the inner cell mass (ICM) of mammalian blastocyst stage embryos. ESCs are able to undergo self-renewing cell division under specific cell culture conditions for extended periods, thereby maintaining their pluripotency (see e.g. Loebel, D. A. et al. (2003) Dev. Biol. 264, 1-14 or Smith, A. G. (2001) Annu. Rev. Cell Dev. Biol. 17, 435-462).

While ESCs can be differentiated in a controlled fashion, for instance into neurons in the presence of nerve growth factor and retinoic acid (Schuldiner et al. (2001) Br. Res. 913, 201-205), their ability to readily differentiate has posed a major practical challenge. In order to maintain ESCs in a pluripotent state, their differentiating during handling and growing in culture has to be prevented. For this reason they are traditionally cultured in the presence of fetal calf serum on a layer of feeder cells (see e.g. U.S. Pat. No. 5,843,780 and No. 6,090,622) or in fibroblast-conditioned medium (CM). Nevertheless, even under carefully controlled conditions ESCs may undergo spontaneous differentiation during in-vitro propagation. Leukemia inhibitory factor (LIF), a factor mediating self-renewal in mouse ESCs, has also been found to inhibit differentiation of mouse ESCs, but it does not replace the role of feeder cells in preventing differentiation of human ESCs. Therefore, means of maintaining pluripotency and/or self-renewing characteristics of ECS would be a substantial achievement towards realizing the full commercial potential of stem cell therapy.

Adult stem cells, although not pluripotent like ESCs, have been shown to be capable of self-renewal and to be of a plasticity rendering their developmental capabilities comparable to those of the more immature pluripotent ESCs. As an example, an adult stem cell is able to differentiate into a cell lineage different from its tissue of origin.

Stem cells are also found in carcinomas called teratoma of various tissue (often of the testes and the ovary) that produce tissues consisting of a mixture of two or more embryological layers. The malignant forms of such carcinomas are also called teratocarcinoma. Development of stem cells in murine teratocarcinomas parallels events in the normal embryo. Their presence can explain that chemotherapy often removes the bulk of a tumor mass without preventing tumor recurrence (Chambers, I. Smith, A, (2004) Oncogene 23, 7150-7160). Therefore, means of abrogating pluripotency and/or self-renewing characteristics of stem cells in such tumors would typically be a precondition for a permanent removal of such carcinomas.

A small number of proteins has so far been identified as being important for the normal development of the pluripotent cells and/or in maintaining the pluripotent cell state. These include the proteins Oct4 (Nichols, J. et al. (1998) Cell 95, 379-391), Sox2 (Avilion, A. A. et al. (2003) Genes Dev. 17, 126-140), Nanog (Mitsui et al (2003) Cell 113, 631-642; Chambers et al (2003) Cell 113, 643-655) and FoxD3 (Hanna et al (2002) Genes Dev. 16, 2650-2651). Other proteins such as LIF (see above) or Rex1 (Zfp42) have also been suggested to be involved in such functions. The mechanisms of their action as well as the specific role that these proteins may play in the development of pluripotent cells or in maintaining a pluripotent cell state are however so far unknown.

Accordingly, it is the object of the present invention to provide a method of controllably maintaining pluripotency and/or self-renewing characteristics of stem/progenitor cells as well as a method of modulating gene expression in a cell, for example in order to abrogate pluripotency and/or self-renewing characteristics of stem cells in teratomas.

### SUMMARY OF THE INVENTION

In one aspect, the invention thus provides a method for maintaining pluripotency and/or self-renewing characteristics of stem/progenitor cells. The method includes contacting at least two transcription factors, or a functional fragment thereof, with the promoter region of the nanog gene. One of the at least two transcription factors is selected from the POU- and homeo-domain-containing transcription factors. Another of the at least two transcription factors is selected from the HMG domain-containing transcription factors. The method further includes allowing the at least two transcription factors to form a complex with a specific binding element within the nanog promoter. The complex thus formed regulates nanog gene expression by mediating transcriptional activation.

In another aspect, the invention provides a method for modulating gene expression in a cell. The method includes contacting at least two transcription factors, or a functional fragment thereof, with the promoter region of the nanog gene. One of the at least two transcription factors is selected from the POU- and homeo-domain-containing transcription factors. Another of the at least two transcription factors is selected from the HMG domain-containing transcription factors. The method further includes allowing the at least two transcription factors to form a complex with a specific binding element within the nanog promoter. The complex thus formed regulates nanog gene expression by mediating transcriptional activation.

### BRIEF DESCRIPTION OF THE DRAWINGS

The invention will be better understood with reference to the detailed description when considered in conjunction with the non-limiting examples and the drawings, in which:

FIG. 1 depicts the effect of Nanog downregulation in embryonic stem cells (ESCs) by means of Nanog RNAi. (A) Specificity of the used Nanog RNAi was tested by co-transfection of Nanog expression vector with constructs expressing control, Nanog, Oct4 or Sox2 siRNA into 293T cells. The cell lysates were analyzed by Western blot with anti-Nanog or anti-β actin antibodies. β actin served as a loading control. (B) Nanog siRNA did not affect Sox2 expression. Nanog or control siRNA constructs were co-transfected with Sox2 expression vector into 293T cells. The lysates were analyzed by Western blot with anti-Sox2 or anti-β actin antibodies. (C) Nanog siRNA did not affect Oct4 expression. Nanog or control siRNA constructs were co-transfected with Oct4 expression vector into 293T cells. The lysates were analyzed by Western blot with anti-Oct4 or anti-β actin antibodies. (D) Nanog knockdown reduced Nanog in ES cells. Nanog or control siRNA construct was transfected into ES cells. The lysates were probed using anti-Nanog or anti-β actin antibodies. (E) Nanog knockdown induced differentiation in ES cells. The Nanog and control knockdown cells were stained for alkaline phosphatase. A photo was taken at the same time of ESCs transfected with control siRNA (I) and ESCs transfected with Nanog siRNA (II). Note the presence of flattened epithelial-like cells in knockdown cells not seen at all in vector control ES cells.

FIG. 2 depicts an electrophoretic mobility shift-assay (EMSA) as an exemplary method to analyse the complex formation of a POU- and homeo-domain-containing transcription factor and a HMG domain-containing transcription factor with a specific binding element within the nanog promoter. The present example illustrates the binding oft Oct4 and Sox2 to the Nanog octamer/HMG composite element. EMSA was performed using either the labeled putative Oct4/Sox2 element from the nanog promoter or the known Oct4/Sox2 binding site from the FGF4 enhancer as a positive control. Binding of factors was tested in crude nuclear extracts of E14 embryonic stem cells. Protein-DNA complexes containing Oct4 and Sox2 were identified by addition of antibodies which supershifted the indicated bands. Binding specificity was tested using oligo competitors (as indicated on top of the depicted PAGE-gel), which indicated that binding of Oct4 required an intact octamer motif and binding of Sox2 required an intact HMG element. Heterodimerization required both be intact. Key to competitors: N=Nanog, F=FGF4, O=Oct4 binding site, S=Sox2 binding site, mO=mutated Oct4 binding site, mS=mutated Sox2 binding site, Gbx=non-specific competitor.

FIG. 3 shows an in vivo interaction of Oct4/OCT4 and Sox2/SOX2 with the Nanog/NANOG promoter. (A) Chromatin immunoprecipitation (ChIP) was performed on undifferentiated mouse ESCs (0 day retinoic acid, RA) and ESCs cultured in the presence of retinoic acid (RA) for 3 and 6 days. Immunoprecipitation with antibodies against Oct4 (N19) and Sox2 (Y17) are shown. Fold enrichment measured by real time PCR, of 3 different amplicons were compared, one of these (amplicons 2) encompasses the composite oct-sox element (open box in schematic) and the two others (amplicons 1 & 3) do not. The position, relative to the transcription start site, of each amplicon is indicated in the schematic diagram and exon 1 of Nanog indicated by a solid black box. (B) OCT4 and SOX2 ChIP of NANOG in human ESCs. The fold enrichment of specific amplicons from undifferentiated cells is indicated. The data illustrate that only the three closest amplicons to the composite sox-oct element showed enrichment with both antibodies used. All standard deviations are indicated.

FIG. 4 shows that the nanog proximal promoter drives EGFP (enhanced green fluorescence protein) expression in undifferentiated ES cells but not in their differentiated derivatives. A mouse ESC line (NanogEGFP) stably transfected with a vector construct containing the nanog promoter (−289 to +117 relative to the transcription start site) driving EGFP expression was compared under different culture conditions. Light (A, C, E) and fluorescence (B, D, F) microscopy are depicted of undifferentiated ESCs (A, B), ESCs cultured without LIF for 3 days (C, D), and ESCs treated with 0.1 μM retinoic acid (RA) for 3 days (E, F). Withdrawal of LIF, a less aggressive induction of differentiation than exposure to RA, reduced EGFP expression noticeably. RA, a strong inducer of ESC differentiation, drastically reduced EGFP expression (G) Real-time PCR detection of transcripts levels of endogenous Nanog compared with that of the Nanog promoter-driven EGFP under undifferentiated and differentiating conditions. Numbers within the graph indicate percentage of EGFP-positive cells as measured by FACS analysis. EB, embryoid body; RA, retinoic acid.

FIG. 5 A illustrates an example of an identification of a binding element by phylogenetic footprinting. nanog sequences were obtained from mouse, rat, human, cow and elephant genome sequencing projects. Positions, in the region −212 to −119 relative to the transcription start site, that remain invariant over >250 million years of cumulative evolution are shaded. The oct-sox composite element is indicated as are the 3 bp replacement mutations with their corresponding names given below.

FIGS. 5 B and C illustrate the analysis of the effect of the complex formation between Nanog and a transcription factor containing a (POU)-specific domain and a homeo-domain as well as a transcription factor containing a HMG domain. Depicted are promoter-luciferase reporter assays of various nanog promoter constructs transiently transfected into F9 teratocarcinoma cells. The luciferase activity of the −289 to +117 mouse nanog promoter fragment (wt) is arbitrarily set at 100%. (B) Activity of altered mouse promoter constructs compared to the wild-type (wt), short (short wildtype promoter, −230 to +63 region), For and Rev are identical to one another but for the conserved region (−93 to +117) in the reverse orientation relative to the transcription start site. Hs.1 and Hs.2 are the homologous regions to the mouse wt construct from human Nanog and NanogGP1, respectively. (C) Effects of replacement mutations on the activity of the mouse promoter, the mutations correspond to those indicated in A with the oct/sox construct having both these elements mutated.

FIG. 6 depicts the results of an RNAi knockdown of Oct4 or Sox2 on Nanog promoter activity. FIG. 2A illustrates schematically the cotransfection of RNAi constructs targeting Sox2, Oct4, and EGFP (as a control) with a Nanog promoter-luciferase reporter construct into mouse ESCs. FIG. 2A shows the luciferase activity measured 3 days after transfection (B). Luciferase activity was measured relative to the Renilla luciferase internal control. A construct without inhibitory RNA was used as a negative control (−). As can be seen, Nanog promoter activity is reduced by RNAi constructs targeting both Sox2 and Oct4. Standard deviations are indicated.

### DETAILED DESCRIPTION

The present invention is based on the surprising findings that transcription factors that contain a POU-specific domain and a homeo-domain and transcription factors that contain a HMG domain form a complex with a promoter of nanog both in vitro and in vivo, and that furthermore the formation of this complex regulates the expression of the protein Nanog. It was even more surprising that at least one such binding region was found to have remained invariant over an accumulated 250 million years of evolution.

The inventors' finding is of particular practical relevance, since a transcription factor that contains a POU-specific domain and a homeo-domain is the protein Oct4, and a transcription factors that contains a HMG domain is the protein Sox2 (cf. above and see also below). Oct4, also known as Oct3, is often used as a marker for the undifferentiated state of cells. Oct4 is highly expressed in human and mouse ESCs, and its expression diminishes when these cells differentiate and lose pluripotency (Palmieri et al. (1994) Dev. Biol. 166, 259-267). It is also expressed in adult stem cells, in tumor cells and in immortalized non-tumorigenic cells, but not in cells of differentiated tissues (see e.g. Monk, M., Holding, C. (2001) Oncogene 20, 8085-8091).

Sox2 is a transcription factor, and like Oct4, it is strongly upregulated in undifferentiated human ESCs, when compared to differentiating ESCs (Brandenberger, R et al. [2004] Nature Biotechnology 22, 707-716). Sox2 is also expressed for instance in the immature, undifferentiated cells of the neural epithelium of the entire CNS in the early stages of embryonic development. Upon differentiation Sox2 is down-regulated (Stevanovic, M [2003] Molecular Biology Reports 30, 2, 127-132). Although both Sox2 and Oct4 have independent roles in determining other cell types (Niwa, H. et al. (2000) Nat Genet. 24, 372-37; Avilion, A. A. et al., supra), at least part of their function in pluripotent cells is via a synergistic interaction between the two to drive transcription of target genes.

The term transcription factor refers to the ability of a protein in altering the level of synthesis of RNA from DNA (transcription). Typically such a factor is a cytoplasmic or nuclear protein which binds to a defined region of a gene found on a respective DNA, such as enhancer elements or promoter elements, thus forming a complex with the DNA.

Three currently known targets of a synergistic action of Oct4 and Sox2 are fgf4, utf1, and fbx15 (Yuan, H. et al. (1995) Genes Dev. 9, 2635-2645; Nishimoto, M. et al. (1999) Mol. Cell. Biol. 19, 5453-5465; Tokuzawa, Y. et al. (2003) Mol. Cell. Biol. 23, 2699-2708), a fourth appears to be sox2 itself (Tomioka, M. et al. (2002) Nucleic Acids Res. 30: 3202-3213) though the corresponding cis-elements appear to be dispensable for pluripotent cell expression (Hayashi, S. et al. (2002) Mech. Dev. 119 Supp1 1, S97-S101). Each of these target genes has a composite element containing an octamer and a sox binding site.

In the methods of the present invention, transcription factors as indicated above (cf. also below) form a complex with a promoter of a gene, which encodes a newly identified protein named Nanog. This is a further protein that is essential in the maintenance of the pluripotent state of cells as well as in the normal development of the pluripotent cells of the early epiblast/inner cell mass (ICM) and the derivation of ES cells from this population of cells (Mitsui, et al., supra). It is also often used as a marker for the undifferentiated state of cells, e.g. stem cells or embryonic germ cells, since it is present much more frequently in for instance undifferentiated ESCs than in differentiating ESCs (Brandenberger, R et al., supra). Nanog is also expressed in adult bone marrow and at a low level in various adult tissues such as renal cells and in certain forms of cancer. Like Oct-4, Nanog is known for its ability to restore some embryonic-like plasticity to mature adult cells (see e.g. Theise, N. L., Wilmut, I [2003] Nature 425, 21).

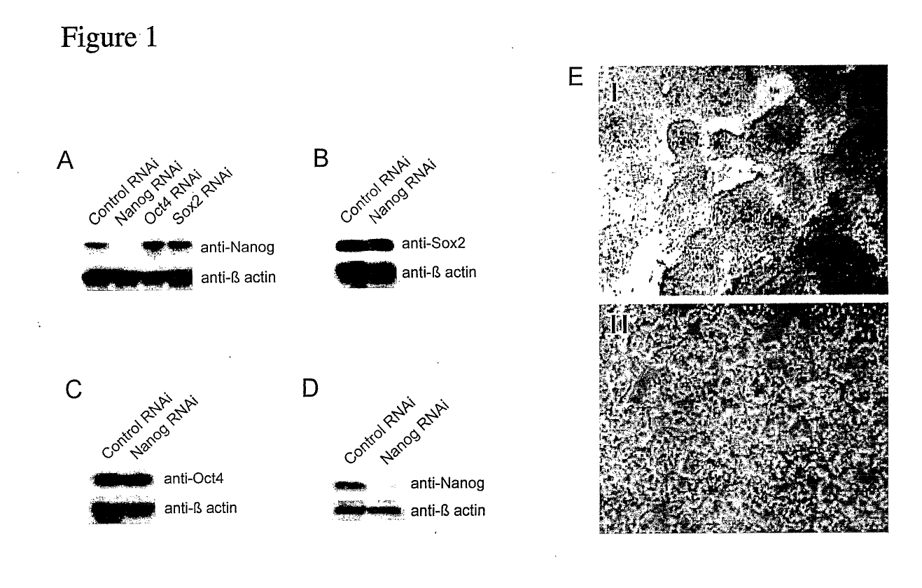

Nanog contains a homeobox and may thus also act as a transcription factor. Over-expression of nanog is capable of maintaining the pluripotency and self-renewing characteristics of ESCs under what normally would be differentiation-inducing culture conditions (Chambers, I. et al., supra). Downregulation of nanog expression under standard growth conditions induces differentiation in ESCs (see FIG. 1). Concomitant with this essential function in pluripotent cell maintenance is its restricted expression pattern. Nanog transcripts first appear in the inner cells of the morula prior to blastocyst formation (Chambers, I. et al., supra; Mitsui, K. et al., supra), and in the blastocyst expression is restricted to the inner cell mass (ICM; Wang, S. H. et al. (2003) Gene Expr. Patterns 3, 99-103), and is no longer detectable at implantation.

In view of the findings by the present inventors the recently published observation that Oct4 and Sox2 have a similar expression pattern through mouse preimplantation development (Avillon, A. A. et al. (2003) Genes Dev. 17, 126-140) becomes easily understandable. Building on the knowledge that Nanog is a key element for maintaining the pluripotent cell state and self-renewing characteristics of stem cells, the inventors were thus able to establish a method of maintaining pluripotency and/or self-renewing characteristics of stem cells and progenitor cells. The inventors have furthermore found their method to be suitable for modulating gene expression in a cell.

The nanog gene encodes the first known transcription factor that appears after compaction and specific to the inner cells of the morula, both Oct4 and Sox2 are expressed prior to compaction in all blastomeres. The term “nanog gene” as used herein shall be understood to include all mammalian nanog genes, such as for instance the mouse and human nanog genes as described for instance in Hart, A. H. et al. ([2004] Dev. Dynamics 230, 187-198), the bovine gene of the NCBI GeneID: 538951, the pig genes of the EMBL accession Nos Q5GMQ0 and Q64HX3, the dingo gene of NCBI GeneID: 486701, the chimpanzee gene of the NCBI GeneID: 452438, the macaque gene of the EMBL accession No Q5TM84, the rat gene of the NCBI GeneID: 414065 (Locus tag: RGD:1303178), the goat gene transcribing RNA of NCBI accession No AY786437 and encoding the transcription factor of NCBI accession No AAW50709, as well as orthologs thereof. It also includes nanog genes yet to be identified. It furthermore includes nanog pseudogenes such as for instance identified by Anne et al. ([2004] Genomics 84, 229-238), where they have been altered by standard methods known in the art (e.g. site directed mutagenesis) to be functional. It furthermore includes forms of the gene that deviate in their nucleic acid sequence, when compared to the known nanog sequence. The difference can for instance be due to a polymorphism, changes or modifications of single nucleotides, substitutions, deletions or insertions (of continuous stretches), and N- and/or C-terminal additions introduced into the natural sequence of the corresponding nucleic acid sequence. The promoter region of the nanog gene used in the method of the present invention may be of, or derived from, any species. Two illustrative examples are the promoter region of the human and of the mouse nanog gene.

As Nanog is expressed in both mouse and human pluripotent cells it can be reasoned that the pluripotent transcription of this gene is maintained through the functional conservation of cis-regulatory elements, and concomitantly, the location and sequence of these elements would be conserved through purifying selection. Therefore mouse and human nanog genomic sequences were derived from the public databases for sequence comparisons. Surprisingly, two copies of the human nanog gene resulting from a tandem duplication of mouse nanog and slc2a3 were identified (Booth, H. A. and Holland, P. W. (2004) Genomics 84, 229-238; Hart, A. H. et al., supra). Based on human EST data (Hs.329296) NANOG/NANOG1 is actively transcribed whereas NANOGP1/NANOG2 remains at undetectable levels. In the examples below actively transcribed NANOG was used to compare it to its mouse homolog nanog.

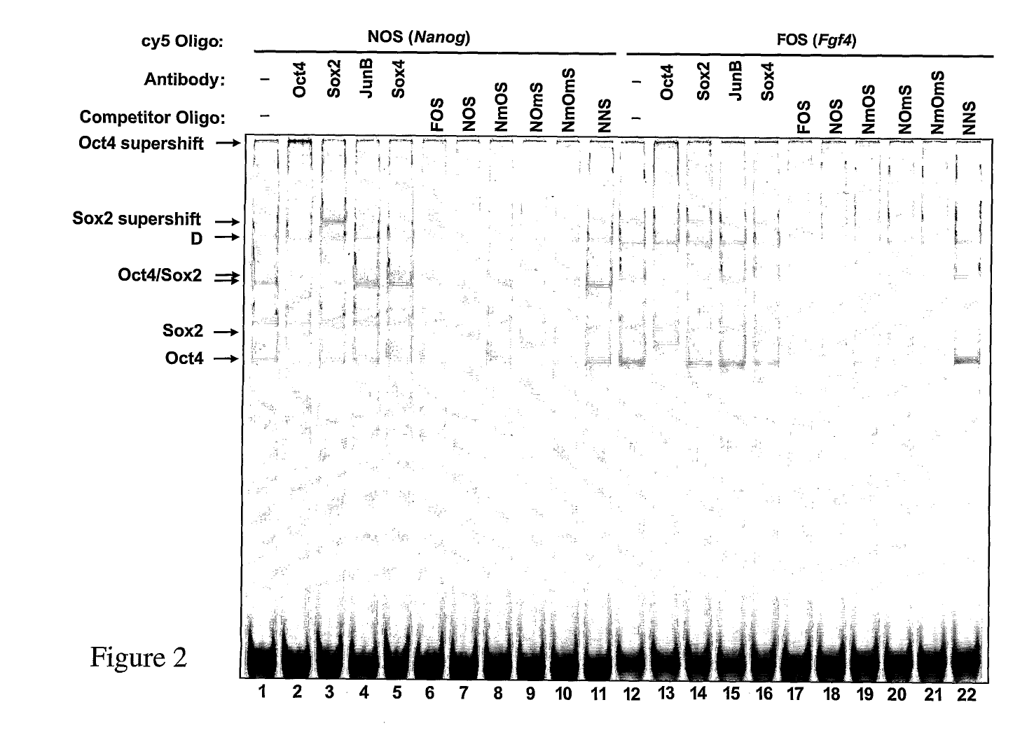

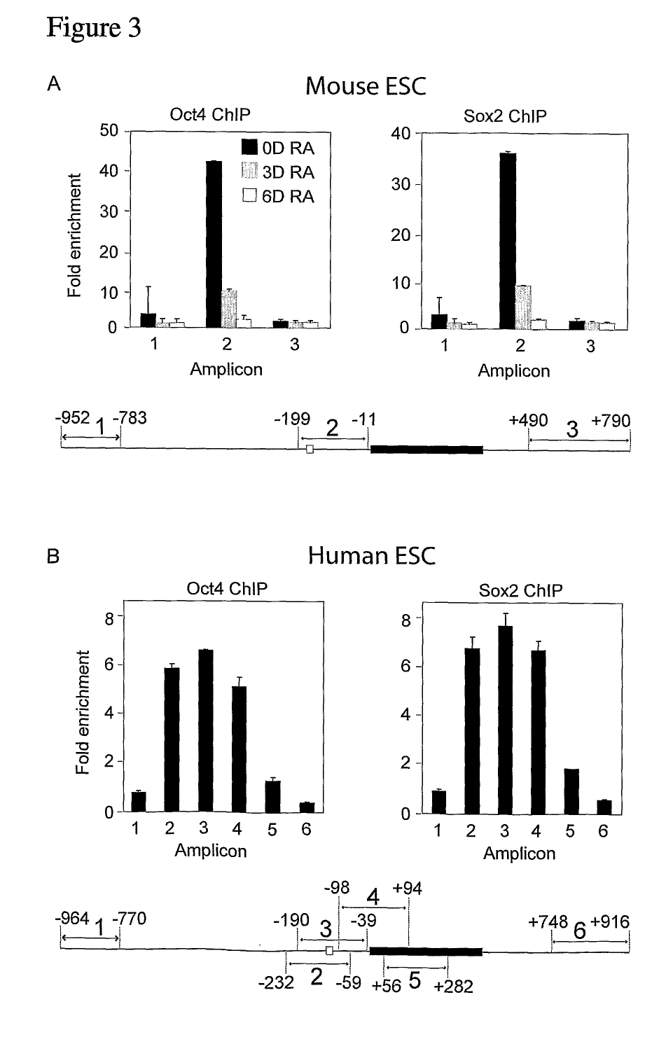

The methods of the present invention include contacting at least two transcription factors, or a functional fragment thereof, with the promoter region of the nanog gene, i.e. the nucleic acid sequence of the nanog gene which is recognized and bound by an RNA polymerase during the initiation of transcription. One of these transcription factors is selected from the transcription factors that contain a Pit, Oct, Unc (POU)-specific domain and a homeo-domain. In many embodiments the homeo-domain is a POU-homeo-domain. A POU-specific domain is generally 75-82 amino acids long, a POU-homeo-domain about 60 amino acids long. In native transcription factors these two domains typically together form the so called POU-domain, wherein the POU-specific domain forms the N-terminal region and the POU-homeodomain the C-terminal region. Although both the POU-specific and the POU-homeodomain are required in the methods of the present invention, they can be selected in any arrangement as long as a complex formation with a promoter of the nanog gene occurs. In many embodiments they are connected by a linker region of a suitable length. Those skilled in the art will be aware that nonconserved sequences outside the POU domain have been reported to participate in the transactivation of target sequences. Therefore, the suitability of artificially generated transcription factors, or a functional fragment thereof, for the methods of the invention will need to be tested in terms of their binding to nanog (cf. below and e.g. Examples 2 or 3, FIG. 2 or FIG. 3). Examples of suitable transcription factors include, but are not limited to, Pit-1, Oct-1, Oct-2, Oct-4, Oct-6, Oct-11, Brn-3, Brn-3A, B and C, hepatocyte nuclear factor-1α and β, retina-derived POU-domain factor-1, sperm 1 POU-domain transcription factor, the rat protein Rov-1, transcription factors POU1, POU2, GBX-2 and Oct-1.5 of Xenopus laevis, transcription factor Cf1a of Drosophila melanogaster, the Ciona intestinalis factor of the NCBI Acc. No. BAE06650, the protein encoded by the C. elegans gene unc-86, the C. elegans factor ceh-6, the Drosophila proteins dPOU-19/pdm-1 and dPOU-19/pdm-2, and the zebrafish factor pou5f1/pou2. As an illustrative example, the transcription factors Oct-1, Oct-4 and Oct-6 have been shown to bind to a region on the nanog promoter (Wu, D, Yao, Z, [2005] Cell Research 15, 5, 317-324).

A second of these at least two transcription factors, or a functional fragment thereof, is selected from the HMG (high mobility group) domain-containing transcription factors. HMG DNA binding domains confer significant preference for distorted DNA, such as 4-way junctions. Examples of such transcription factors include, but are not limited to, Sry HMG box (Sox) transcription factors, HrSoxB1, the lymphoid enhancer-binding factor-1 (LEF-1, T cell-specific transcription factor 1-α), HMGB1a protein, HMGB1b protein, upstream binding factors (UBFs), CCAAT-binding transcription factor 2 (CTF2), activating transcription factor 4 (ATF4), proteins of the YABBY family, mitochondrial transcription factor A (mtTFA), granulosa cell high-mobility group-box protein-1 (GCX-1), T-cell specific transcription factor 7, Prf1 of the smut fungus Ustilago maydis, the factor Ste11p and the unnamed factor with NCBI Acc. No NP 595672 of Schizosaccharomyces pombe, TCF/LEF-1 of C. elegans, XTcf3 of Xenopus, the testis-determining factor SRY, XSOX3, the transcription product of sea urchin gene Unichrom, Mating-type protein A2 from Kluyveromyces lactis, the protein CG17964-PH isoform H (NCBI Acc. No NP 001014685) of Drosophila melanogaster, and the transcription factor with NCBI Acc. No T12113 of fava bean.

In some embodiments the second transcription factor is thus a SOX (SRY-related HMG box) protein, such as, but not limited to, Sox-1, Sox-2, Sox-3, Sox-4, Sox6, Sox7, Sox8, Sox9, Sox10, Sox11, Sox-13, Sox14, Sox15 Sox18, Sox20, Sox21, Sox30, Sox32 or the factor Sox-11-D of Xenopus laevis, or a functional fragment thereof.

A functional fragment of one of the respective transcription factors may be of any length, and shall be defined by two criteria. Firstly, a functional fragment is able to bind to and form a complex with a binding region of the nanog promoter that is stable enough to affect the activity of a respective gene driven by this promoter, e.g. the nanog gene. Secondly, such a functional fragment may have at least 60% sequence identity with the corresponding amino acid sequence of a naturally existing transcription factor that contains a POU- and a homeo-domain (e.g. a POU-homeo-domain), or a HMG domain respectively. In some embodiments, a respective fragment has at least 80%, and in some embodiments at least 95% sequence identity with the corresponding amino acid sequence of a known respective transcription factor. The term “sequence identity” refers to the percentage of pair-wise identical residues obtained after a homology alignment of an amino acid sequence of a known POU- and homeo-domain-containing or HMG domain-containing transcription factor, respectively, with an amino acid sequence in question, wherein the percentage figure refers to the number of residues in the longer of the two sequences.

A functional fragment of a transcription factor used in a method of the present invention may furthermore be merged with additional sequences, for instance in form of a fusion protein. It may also include natural or artificial chemical modifications, such as for instance a so called affinity tag or a label. Examples of affinity tags include, but are not limited to biotin, dinitrophenol, digoxigenin, oligohistidine, polyhistidine, an immunoglobulin domain, maltose-binding protein, glutathione-S-transferase (GST), calmodulin binding peptide (CBP), FLAG′-peptide, the T7 epitope (Ala-Ser-Met-Thr-Gly-Gly-Gln-Gln-Met-Gly), maltose binding protein (MBP), the HSV epitope of the sequence Gln-Pro-Glu-Leu-Ala-Pro-Glu-Asp-Pro-Glu-Asp of herpes simplex virus glycoprotein D, the hemagglutinin (HA) epitope of the sequence Tyr-Pro-Tyr-Asp-Val-Pro-Asp-Tyr-Ala and the “myc” epitope of the transcription factor c-myc of the sequence Glu-Gln-Lys-Leu-Ile-Ser-Glu-Glu-Asp-Leu. A label may additionally be a moiety that assists in detection, where this is for instance desired in the method of the present invention. Examples include, but are not limited to, a radioactive amino acid, fluorescein isothiocyanate, 5,6-carboxymethyl fluorescein, Texas red, nitrobenz-2-oxa-1,3-diazol-4-yl, coumarin, dansyl chloride, rhodamine, amino-methyl coumarin, Eosin, Erythrosin, BODIPY®, Cascade Blue®, Oregon Green®, pyrene, lissamine, xanthene, acridine, oxazines, phycoerythrin, Cy3, Cy3.5, Cy5, Cy5.5, and Cy7 enzymes. Further suitable enzymes include, but are not limited to, alkaline phosphatase, soybean peroxidase, or horseradish peroxidase.

A respective affinity tag or label may be located within or attached to any part of a selected transcription factor. As an illustrative example, it may be operably fused to the amino terminus or to the carboxy terminus of any POU- and homeo-domain-containing transcription factor or HMG domain-containing transcription factors.

The methods of the present invention further includes allowing the at least two transcription factors (cf. above), or a functional fragment thereof, to form a complex with a specific binding element within the nanog promoter.

In some embodiments of the methods of the invention, the at least two transcription factors, or a functional fragment thereof, form a heterotrimeric complex on the nanog promoter. The formation of a respective complex can be detected by various analytic methods well known to those skilled in the art, such as for example surface plasmon resonance (e.g. Biacore®-technology), nuclear magnetic resonance or crystallization and subsequent X-ray analysis. In some embodiments of the methods of the present invention the transcription factors as elaborated above, or functional fragments thereof, form a heterodimer. This heterodimer forms then a heterotrimeric complex with the nucleic acid that includes the binding region of the nanog promoter. An example of a respective dimer that forms a heterotrimeric complex is the binding of Oct4 and Sox2 to nanog. Crystallographic data indicate that these two and related transcription factors form ternary complexes with binding elements on DNA (Reményi et al. [2003] Genes & Development 17, 2048-2059). A homology model generated using Oct4 onto the Oct1 structure shows that the complex formed between Oct4 and Sox2 resembles the complex between Oct1 and Sox2. Sox2 has two surface patches for interaction with Oct4. One of these is the C terminus of the HMG domain of Sox2, which has also been shown to interact with the DNA. In view of the fact that complex formations between various SOX and POU transcription factors have been well characterized (cf. e.g. Reményi et al., supra), it can be expected that various transcription factors containing POU- and homeo-domains, and such factors containing a HMG domain form a heterotrimeric complex with a binding region on a nucleic acid, such as the nanog gene.

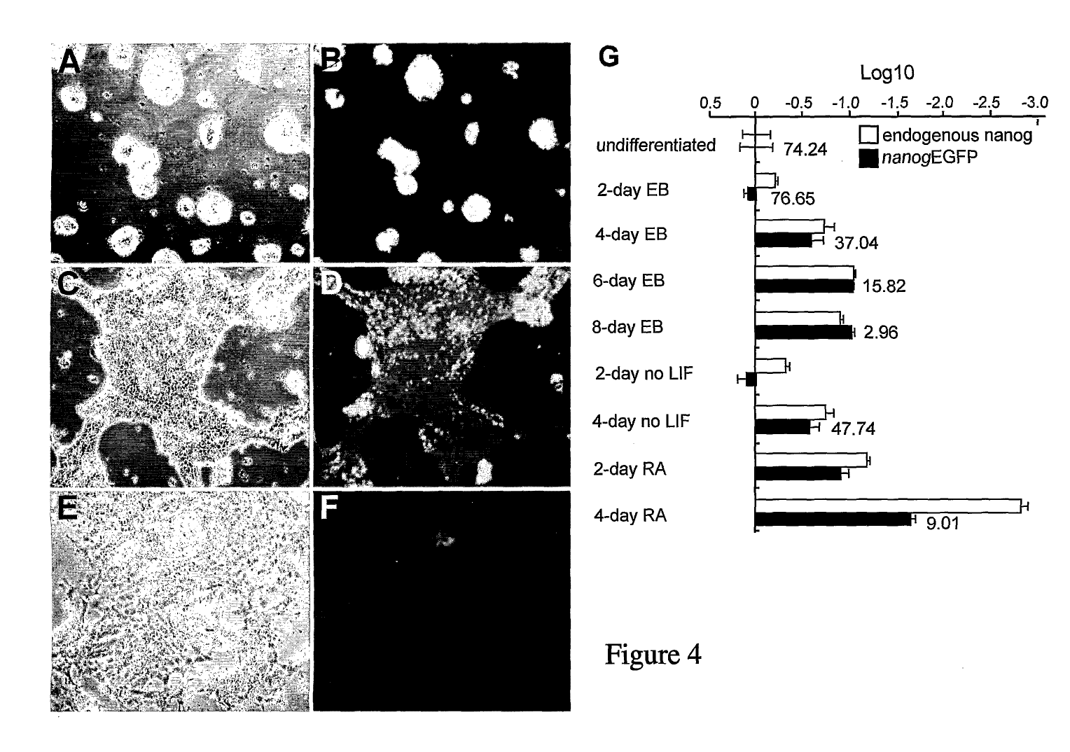

A respective binding element can be identified by various means known to those skilled in the art. Example 4 illustrates an identification of a binding element on NANOG by means of sequence comparisons. Such an approach will typically be performed where no information is available at all, whether a binding element for a respective transcription factor exists. Since the present inventors have already identified at least one such binding element, it will generally not be necessary to use this procedure. An alternative means are computer-implemented methods as disclosed in U.S. Pat. No. 6,735,530, for example. Yet a further means of identifying a binding element is a chromatin immunoprecipitation assay, as for instance described in Example 3. This method can furthermore be used to determine regions of active transcription, or to assess modifications of genome structure by histone-binding. Yet another means is an electrophoretic mobility shift-assay (EMSA) as for example described in Example 2 and FIG. 2. A further method of identifying a binding element in the nanog promoter is the use of the promoter for the expression of a marker, such as for instance of a fluorescent protein. An example how this approach may be put into practice is indicated in FIG. 4. Mouse ESC lines were transfected with a plasmid vector containing the mouse sequence from −289 to +117 of the nanog gene (relative to the transcription start site (TSS)) driving the expression of an enhanced green fluorescent protein (EGFP). Induction of differentiation by retinoic acid downregulated the nanog gene and thus drastically reduced EGFP expression after 3 days of treatment (cf. FIG. 4E,F). A comparison of NanogEGFP to that of the endogenous Nanog itself by means of realtime PCR (FIG. 4G) showed that the construct was able to recapitulate endogenous Nanog expressions. Those skilled in the art will be aware that these methods can furthermore be combined with other methods such as in vitro mutagenesis to further verify certain regions as being part of a binding element.

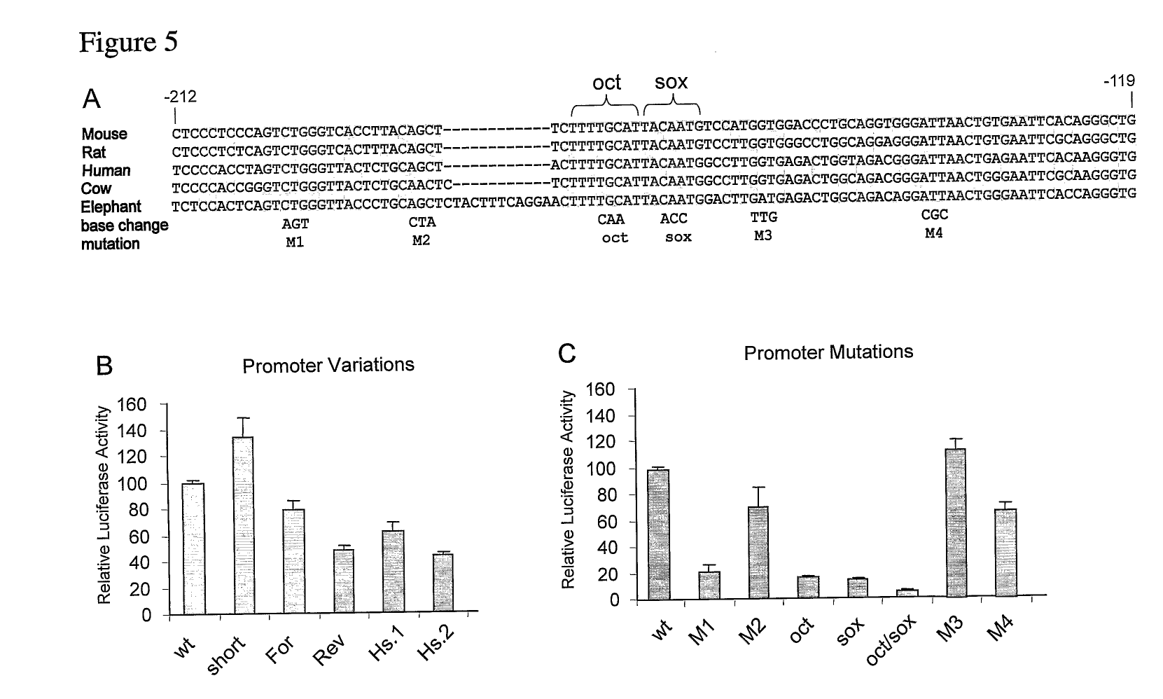

In some embodiments of the invention the at least two transcription factors bind to a composite element within the nanog promoter. Thus, in these embodiments the POU- and homeo-domain-containing transcription factor binds to one part of the composite element, and the HMG domain-containing transcription factor binds to another part of the composite element. The respective binding element may include any number of binding regions for each respective transcription factor. It may for instance be a bipartite binding site. Such binding elements are for example known to exist for the two transcription factors Sox2 and Oct4. Genes that contain a composite element containing an octamer and a sox binding site and being targeted by Sox2 and Oct4 are Fgf4, Utf1, Fbx15 and Sox2 and Pou5f1 (the gene encoding Oct4), themselves (Yuan, H., et al, supra; Nishimoto, M., et al., supra; Tokuzawa, Y., et al, supra; Tomioka, M., et al, supra; Chew, J.-L., et al. (2005) Mol. Cell. Biol. 25, 14, 6031-6046; Okumura-Nakanishi, S. et al. (2005) J. Biol. Chem. 280, 5307-5317). The inventors have identified a respective composite element in the nanog promoter (cf. e.g. FIG. 5A). In some embodiments the composite binding element is thus an oct4/Sox2 binding site. Within the oct4/Sox2 binding site the binding regions for oct4 and for Sox2 may be arranged in any orientation, and in any location with respect to each other as long as both Sox2 and oct4 are able to simultaneously bind to the composite binding site. The two regions may for instance be immediately adjacent to each other (see FIG. 5A).

In some embodiments of the invention the formation of the complex between the binding element within the promoter region of the nanog gene and the at least two transcription factors, or a functional fragment thereof, is furthermore being detected. Any method may be used for this purpose that is sensitive enough to detect the binding of a protein to a nucleic acid. Such methods may for instance rely on spectroscopic, photochemical, photometric, fluorometric, radiological, enzymatic or thermodynamic means, or on cellular effects. An example for a spectroscopic detection method is fluorescence correlation spectroscopy (Thompson, N. L. et al. [2002] Curr. Opin. Struct. Biol. 12, 5, 634-641). A photochemical method is for instance photochemical cross-linking (Steen, H., Jensen, O. N. [2002] Mass. Spectrom. Rev. 21, 3, 163-182). The use of photoactive, fluorescent, radioactive or enzymatic labels respectively (for an overview see: Rippe R. A. et al. [2001] Methods Mol Biol. 160, 459-479) are examples for photometric, fluorometric, radiological and enzymatic detection methods. An example for a thermodynamic detection method is isothermal titration calorimetry (ITC, for an overview see: Velazquez-Campoy, A. et al. [2004] Methods Mol Biol. 261, 35-54). Any cellular effect, for instance a change in phenotype, may be caused by the expression of a recombinant factor under the control of the promoter region of the nanog gene. An example of a method using cellular effects may also include the measurement of the cell differentiation status (cf. e.g. Noaksson, K. et al. [2005] Stem Cell Express doi: 10.1634/stemcells.2005-0093), including determining its pattern of marker proteins. Some of these methods may include additional separation techniques such as electrophoresis or HPLC. In detail, examples for the use of a label include, but are not limited to, a compound as a probe or an immunoglobulin with an attached enzyme, the reaction catalysed by which leads to a detectable signal. An example of a method using a radioactive label and a separation by electrophoresis is an electrophoretic mobility shift assay.

Hence, examples of detecting the formation of the complex between the binding element of the nanog gene and the at least two transcription factors, or a functional fragment thereof, also include, but are not limited to, examples that are suitable for the identification of the binding element such as an immune precipitation (or Western blot hybridization) or a chromatin immunoprecipitation assay, an electrophoretic mobility shift-assay, or surface plasmon resonance.

In some embodiments the region of the respective nanog gene to which the at least two transcription factors bind, is located within the region that corresponds to the region of the human nanog gene that includes sequence positions −289 to +117 of the human nanog gene. In one embodiment this region corresponds to the region of the human nanog gene that includes sequence positions −212 to −119 of the human nanog gene.

As elaborated above, in the methods of the present invention a complex is formed between the at least two transcription factors, or a functional fragment thereof, and a binding element within the promoter region of the nanog gene. The formation of this complex furthermore regulates the nanog gene expression. This effect is achieved by mediating transcriptional activation. Thus, the binding of the at least two transcription factors increases the transcription of the nanog gene. An increased transcription of the nanog gene has been found to maintain a pluripotent cell state of ESCs (supra). A suppression of the transcription of the nanog gene has been found to induce differentiation in human ECs and human embryonic carcinoma cells (cf. FIG. 1 E and Hyslop, L. et al. [2005] Stem Cell Express doi: 10.1634/stemcells.2005-0080).

In some embodiments of the invention the increase in the transcription of the nanog gene is measured in terms of the nanog gene expression. This can for instance be achieved by determining the number of RNA molecules transcribed from a gene that is under the control of the nanog promoter. A method commonly used in the art is the subsequent copy of RNA to cDNA using reverse transcriptase and the coupling of the cDNA molecules to a fluorescent dye. The analysis is typically performed in form of a DNA microarray. Numerous respective services and kits are commercially available, for instance GeneChip® expression arrays from Affymetrix. Other means of determining nanog gene expression include, but are not limited to, oligonucleotide arrays, and quantitative Real-time Polymerase Chain Reaction (RT-PCR).

Example 5 illustrates a further means of determining nanog gene expression, the use of a luciferase reporter vector. In this method expression levels are reflected by luciferase activity of cells expressing a vector comprising the nanog promoter. Luciferase activity can be detected in a luminometer using commercially available kits (see e.g. Example 5).

In some embodiments it may be advantageous or desired to calibrate nanog gene expression data or to rate them. Thus, in some embodiments the methods of the invention additionally include the comparison of obtained results with those of one or more control measurements.

Such a control measurement may include any condition that varies from the main measurement itself. It may include conditions of the method under which for example no expression under the control of the nanog promoter occurs or under which a complex formation between the transcription factors and the nanog promoter cannot occur or cannot be modulated. In some embodiments it may include the use of a compound that adjusts the activity of the nanog promoter to a defined level. In other embodiments a respective compound may prevent the complex formation of the nanog promoter and one of the two transcription factors.

A further means of a control measurement is the use of a mutated nanog promoter, which is not able to bind one or both above mentioned transcription factors, or which binds to them with lower or higher affinity of known degree. FIG. 5C shows an example of an identification of respective sites that may be used for mutations. Three sites when mutated had a drastic effect on the function of the pluripotent promoter dropping luciferase activity to 20% or below of the wild-type construct (cf. FIG. 5C). This included the mutations effecting the oct and sox sites.

In some embodiments of the methods of the present invention the two transcription factors act synergistically to activate the transcription of the nanog gene. A respective synergistic action of the two transcription factors can be assessed by comparing data obtained by the action of each individual transcription factor on the transcription of the nanog gene to the action of two (or more) transcription factors on the transcription in combination. Any method that is suitable for determining gene expression, for example those mentioned above, can be employed for this purpose. As an illustrative example, FIG. 5C shows the detection of the nanog gene expression by means of a luciferase activity assay (see above). When both the oct and sox site were mutated in the same construct this further dropped activity to 6% of wild-type, as compared to an activity to about 20% (compared to the wild-type construct).

The method for maintaining pluripotence and/or self-renewing characteristics of the present invention is suitable for any stem cell, progenitor cell, teratoma cell or any cell derived therefrom as long as it is able to express a transcription factor containing a POU- and a POU-homeo domain and a transcription factor containing a HMG domain, which are able to form a complex with a binding element of the nanog promoter. As an illustrative example, any pluripotent human ESC or a respective cell line may be used in the respective method. Means of deriving a population of such cells are well established in the art (cf. e.g. Thomson, J. A. et al. [1998] Science 282, 1145-1147 or Cowan, C. A. et al. [2004] N. Engl. J. Med. 350, 1353-1356). Furthermore, 71 independent human ESC lines are for example known to exist, of which 11 cell lines are available for research purposes (see e.g. the NIH Human Embryonic Stem Cell Registry at http://stemcells.nih.gov/research/registry/eligibilityCriteria.asp), such as GE01, GE09, BG01, BG02, TE06 or WA09. Adult stem cells may for instance be isolated from blood from the placenta and umbilical cord left over after birth, or from myofibers, to which they are associated as so called “satellite cells” (Collins, C. A. et al. [2005] Cell 122, 289-301, see also Rando, T. A. [2005] Nature Medicine 11, 8, 829-831).

Where the method is intended to be used for a progenitor cell, i.e. a cell giving rise to mature somatic cells, any progenitor cell may be used in this method of the invention. Examples of suitable progenitor cells include, but are not limited to, neuronal progenitor cells, endothelial progenitor cell, erythroid progenitor cells, cardiac progenitor cells, oligodendrocyte progenitor cells, retinal progenitor cells, or hematopoietic progenitor cells. Methods of obtaining progenitor cells are well known in the art. As two illustrative examples, a method of obtaining megakaryocyte progenitor cells has been disclosed in US patent application 2005/0176142 and a method of obtaining mouse liver progenitor cell lines has been described by Li et al. ((2005) Stem Cell Express, doi:10.1634/stemcells.2005-0108). Although limited data are so far available, progenitor cells appear to express POU- and homeo-domain-containing transcription factors as well as HMG domain-containing transcription factors in a manner resembling stem cells. Progenitor cells, such as endothelial progenitor cells obtainable from peripheral blood, have for example been found to posses high expression levels of Nanog and Oct-4 (Romagnani, P. et al. [2005] Circ. Res. 97, 314). CNS progenitor cells such as retinal progenitor cells have been reported to express high levels of Sox2 (Graham, V. et al. [2003] Neuron 39, 5, 749-765; Klassen, H. et al. [2004] J. Neurosci. Res. 77, 3, 334-343).

As indicated above, one method of the present invention is a method for modulating gene expression in a cell. Any cell may be used that expresses the above described at least two transcription factors and that includes a functional nanog gene. In some embodiments an endogenous nanog gene is functionally active. In some of these embodiments the respective cell is a stem or a progenitor cell. Examples of stem cells that may be used in the method of the present invention include, but are not limited to, embryonic stem cells, trophoblast stem cells and extraembryonic stem cells. In some embodiments of the methods of the invention an ESC (embryonic stem cell), such as an ESC of human origin, i.e. a human ESC may thus be used. In other embodiments the cell is a progenitor cell (cf above). In yet other embodiments the cell is a cancer cell. An illustrative example of a cancer cell is teratoma cancer cell, such as for example F9, NTERA2, C3H, TES-1, 1246 (including 1246-3A), SuSa (including SuSa/DXR10 and SKOV-3/DXR10), AT805 (including ATDC5), HTST, HGRT, PC (e.g. PCC3/A/1) or GCT27. Two further illustrative example of a cancer cell are a HeLa cell and an MCF-7 cell. In some embodiments the cell is a hybrid cell of a stem cell and a somatic cell. The nanog gene has been shown to be functionally active in such hybrid cells (cf. e.g. Hatano et al. [2005] Mech. Dev. 122, 67-79). In other embodiments the endogenous nanog gene is functionally inactive. In some of these embodiments any cell of an established eukaryotic cell line is selected, such as for instance HEK, COS, CHO, CRE, MT4, DE (duck embryo), QF (quail fibrosarcoma), NS0, BHK, Sf9, PC12, or High 5. An illustrative example is a HEK 293T cell. In yet further embodiments an exogenous nanog gene is introduced by means of recombinant technology, for instance by means of a vector carrying the nanog gene (cf. also below).

In some embodiments the selected transcription factors, or a functional fragment thereof, are endogenously expressed in amounts that are sufficient for the performance of the present method of the invention. In other embodiments the selected transcription factors are largely or entirely absent from the proteome of the cell. In either case the respective transcription factors, or a functional fragment thereof, may be introduced into the cell by means of one or more recombinant vectors that include the genes encoding the desired transcription factors. Where a cell already endogenously expresses the transcription factors of interest, it may in some embodiments be desired to increase the amount of the respective transcription vector in the cell, for instance for the purpose of improving the signal/noise ratio in screening assays. As an illustrative example, Oct4 and other transcription factors (including nanog) have been expressed in ESCs by means of adenovirus vectors (Kawataba, K. et al. [2005] Mol. Therapy 12, 3, 547-553). Typically, but not necessarily, the respective genes will be under the control of an active promoter or of a promoter that can be conveniently activated by external stimuli.

Where it is desired to include further copies of the nanog promoter or the complete nanog gene, i.e. in addition to the endogenous gene of the respective cell, into a cell of interest, this may likewise be achieved by means of a recombinant vector (e.g. Kawataba, K et al., supra). It may also be advantageous to introduce a vector that contains the nanog promoter into the cell for the purpose of facilitating the activation of a nanog promoter (whether of endogenous or exogenous origin). In addition, it may be desired to employ the nanog promoter to express an exogenous gene, for instance to obtain a protein. In this case typically a vector will be chosen that includes the desired exogenous gene under the control of the nanog promoter. For this purpose the sequence of any desired gene may be included into a respective vector. Persons skilled in the art will be aware that it may be required to coexpress additional enzymes in a case where it is desired that a respectively transcribed protein also undergoes posttranslational modifications within the cell used in the method of the present invention.

Examples of exogenous genes that may be used in the present method as being under the control of the nanog promoter include, but are not limited to, a reporter gene, a drug resistance gene, an apoptosis gene (so-called “death” gene) or any other gene with desirable expression in a respective cell. In some embodiments a respective gene may also encode a protein of interest. In such a case the method of the invention may be used to express and obtain the respective protein. Where the gene under the control of the nanog promoter is an apoptosis gene, the present method may for example be used to eliminate pluripotent cells from a tissue.

As an illustrative example, a vector containing an apoptosis gene under the control of the nanog promoter may be introduced into cells of a tissue. The tissue may have been obtained by differentiation of pluripotent cells, such as stem/progenitor cells.

The present method of the invention may then be used to eliminate any remaining undifferentiated, e.g. pluripotent cells from the respective tissue. In this embodiment the present method may be used to prevent such undifferentiated cells from being engrafted into an organism. This embodiment of the present method may therefore for instance be employed to prevent the occurrence of teratomas after transplantation or implantation. As a further illustrative example, a vector containing a drug resistance gene (e.g. an antibiotic restance gene) driven by the nanog promote may be used for stem/progenitor cell selection.

In other embodiments the present method further includes introducing a compound into the respective cell that modulates the complex formation of the above described at least two transcription factors, or a functional fragment thereof, with the binding element of the nanog promoter. In some of these embodiments, the method further includes contacting a transcription factor or transcription factors, a heterodimeric complex thereof, or the binding element of the nanog promoter with such a compound (i.e. that modulates the complex formation of the at least two transcription factors with the binding element of the nanog promoter).

In some of these embodiments the present method is an in-vitro method for the identification of suitable compounds that modulate the formation of the above described complex between the at least two transcription factors or a functional fragment thereof and the binding element on the nanog promoter. In one embodiment the method of the invention is thus used as a screening method for the purpose of identifying or selecting such compounds. Such a screening method may include the simultaneous screening of compound libraries on multiple-well microplates (e.g. conventional 48-, 96-, 384- or 1536 well plates) using automated work stations. As an illustrative example, it may be desired to identify a compound that inhibits the formation of a complex between the binding region within the nanog promoter and the at least two transcription factors as described above. Such a compound may for instance be desired to initiate or assist cell differentiation, for example to target a germ cell tumor or to generate a homogenous population of cells of a certain tissue type. It may also be desired to use such a compound for removing or differentiating a cell. This property may for instance be due to an inhibition of the complex formation of the above described at least two transcription factors, or a functional fragment thereof, and the respective binding element of the nanog promoter (cf. above).

In other embodiments the present method is an in-vivo method and the cell used in the method is part of or included in a mammal or invertebrate species, or in a microorganism. Examples of mammals that may be used include, but are not limited to, a rat, a mouse, a dingo, a cow, a pig, a goat, a chimpanzee, a macaque, and a human. A respective in-vivo method may include administering a compound that modulates the formation of the above described complex between the at least two transcription factors and the binding region within the promoter of the nanog gene.

In further embodiments the present method includes the use of a compound that has been found to modulate the complex formation between a binding element within the nanog promoter and the above described transcription factors, or a functional fragment thereof, in a mammal (cf. e.g. above) or an invertebrate species. In typical embodiments such a use includes the manufacture of a medicament or a pharmaceutical composition that can be administered to for instance a mammal, such as a human.

A compound that modulates the above described complex formation can be administered by any suitable means. If the cell is included in or part of a mammal, the compound may be administered parenterally or non-parenterally (enterally). In a typical embodiment for administering to a mammal, the application ensures a delivery to blood and liver, for instance by administering a preparation of the compound orally, intravenously or by inhalation. Examples for preparations for an oral application are tablets, pills or drinking solutions, examples for preparations for intravenous administrations are injection or infusion solutions, examples of preparations for administration by inhalation are aerosol mixtures or sprays. If the cell is included in or part of a microorganism or used as an individual cell (e.g. a recombinant cell in culture), examples of administration are the injection or addition of the compound to the environment of the cell. In case of the use of an individual cell, the latter form of administration may possibly be performed in combination with a technique that modifies the microorganism. Such a technique may include electroporation or a permeabilisation of the cell membrane.

An in-vivo method of the invention, or the use of a compound for the modulation of the complex formation between a binding element within the nanog promoter and the above described transcription factors, may be used for various purposes. Examples of such purposes are therapeutic, diagnostic or test purposes. In case of a test purpose some methods may include the application of a compound that has already been identified as being able to modulate the complex formation of the above described at least two transcription factors and the binding region within the promoter of the nanog gene, while other methods may be directed at the identification of such compounds. An illustrative example of a therapeutic purpose is the treatment of teratoma.

As already indicated above, the present method of the invention may furthermore be used both in vitro and in vivo to initiate or to assist the differentiation of for instance stem/progenitor cells.

The compound used to modulate the above described complex formation can be of any nature. It may for instance be isolated from a biological or non-biological source or chemically or biotechnologically produced. Examples for such compounds include, without being limited to, small organic molecules or bioactive polymers, such as polypeptides, for instance immunoglobulins or binding proteins with immunoglobulin-like functions, or oligonucleotides.

Exemplary embodiments of a respective compound are a molecule that alters the methylation status of nucleic acids, or a compound that modulates the methylation status of the promoter of a transcription factor containing a POU- and a homeo-domain and/or a transcription factor containing a HMG domain. As an illustrative example, the demethylating molecule 5-azacytidine has been found to increase the expression of Sox2 and Oct4 (Tsuji-Takayama et al. [2004] Biochem. Biophys. Res. Commun. 323, 86-90).

Another embodiment of a compound used to modulate the above described complex formation is a molecule that alters the pattern of posttranslational modifications of at least one of the transcription factors participating in the complex. The activity of transcription factors is known to be regulated by several posttranslational modifications, including phosphorylation, acetylation, or ubiquitylation.

A further embodiment of such a compound is a nucleic acid molecule. The term “nucleic acid molecule” as used herein refers to any nucleic acid in any possible configuration, such as single stranded, double stranded or a combination thereof. Nucleic acids include for instance DNA molecules (e.g., cDNA or genomic DNA), RNA molecules (e.g., mRNA), analogues of the DNA or RNA generated using nucleotide analogues or using nucleic acid chemistry, and PNA (protein nucleic acids). DNA or RNA may be of genomic or synthetic origin and may be single or double stranded. In the present method of the invention typically, but not necessarily, an RNA or a DNA molecule will be used. Such nucleic acid can be e.g. mRNA, cRNA, synthetic RNA, genomic DNA, cDNA synthetic DNA, a copolymer of DNA and RNA, oligonucleotides, etc. A respective nucleic acid may furthermore contain non-natural nucleotide analogues and/or be linked to an affinity tag or a label (cf. above).

Many nucleotide analogues are known and can be used in nucleic acids and oligonucleotides used in the present method of the invention. A nucleotide analogue is a nucleotide containing a modification at for instance the base, sugar, or phosphate moieties. Modifications at the base moiety include natural and synthetic modifications of A, C, G, and T/U, different purine or pyrimidine bases, such as uracil-5-yl, hypoxanthin-9-yl, and 2-aminoadenin-9-yl, as well as non-purine or non-pyrimidine nucleotide bases. Other nucleotide analogues serve as universal bases. Universal bases include 3-nitropyrrole and 5-nitroindole. Universal bases are able to form a base pair with any other base. Base modifications often can be combined with for example a sugar modification, such as for instance 2′-O-methoxyethyl, e.g. to achieve unique properties such as increased duplex stability.

In some embodiments of the present method of the invention the nucleic acid molecule is an aptamer, a Spiegelmer® (described in WO 01/92655), a micro RNA (miRNA) molecule, a short hairpin RNA (shRNA) molecule or a small interfering nucleic acid (siNA) molecule, such as a small interfering RNA (si-RNA) molecule or a repeat-associated small interfering RNA (rasiRNA) molecule.

The use of small interfering RNAs has become a tool to “knock down” specific genes. It makes use of gene silencing or gene suppression through RNA interference (RNAi), which occurs at the posttranscriptional level and involves mRNA degradation. RNA interference represents a cellular mechanism that protects the genome. SiRNA molecules mediate the degradation of their complementary RNA by association of the siRNA with a multiple enzyme complex to form what is called the RNA-induced silencing Complex (RISC). The siRNA becomes part of RISC and is targeted to the complementary RNA species which is then cleaved. This leads to the loss of expression of the respective gene (for a brief overview see Zamore, P D, Haley, B [2005] Science 309, 1519-1524). This technique has for example been applied to silencing parasitic DNA sequences, such as the cleavage of HIV RNA, as disclosed in US patent application 2005/0191618.

A typical embodiment of such a siRNA for the current invention includes an in vitro or in vivo synthesized molecule of 10 to 35 nucleotides, in some embodiments 15 to 25 nucleotides. Two illustrative examples of nucleic acid sequences that may be used to generate a si-RNA molecule by means of transcription (e.g. using the transcription machinery of a living cell) are SEQ ID NO: 31 and SEQ ID NO: 32 (cf. Example 6). A respective si-RNA molecule may be directly synthesized within a cell of interest (including a cell that is part of a microorganism and an animal). It may also be introduced into a respective cell and/or delivered thereto. An illustrative example of delivering a siRNA molecule into selected cells in vivo is its non-covalent binding to a fusion protein of a heavy-chain antibody fragment (Fab) and the nucleic acid binding protein protamin (Song, E. et al. [2005], Nature Biotech. 23, 6, 709-717). In an embodiment of the present invention siRNA molecules are used to induce a degradation of mRNA molecules encoding one or more POU- and homeo-domain-containing transcription factors and/or one or more HMG domain-containing transcription factors (cf. e.g. Example 6).

The methods of the invention may include the use of additional transcription factors that are able to specifically bind to a region on the nanog gene. In some embodiments the methods therefore include contacting at least a third transcription factor with the nanog gene. Examples of such a transcription factor include, but are not limited to members of the SMAD protein family (e.g. SMAD 1, SMAD 2, SMAD 3, SMAD 4, SMAD 5, SMAD 7, and SMAD 9, or SmadA and SmadB from the fox-tapeworm Echinococcus multilocularis), members of the AP1 (Activator protein 1) family, a hand 1 (heart- and neural crest derivatives-expressed protein 1) transcription factor or a hand 1 related transcription factor.

The invention is further illustrated by the following non limiting examples.

### EXAMPLES

### Sequence Analysis & Promoter Constructs

All sequences were found through publicly available data bases at www.ncbi.nlm.nih.gov and/or www.ensembl.org. Bos taurus and Loxodonta africana Nanog promoter regions were constructed from trace file sequences available at NCBI. The web-based (www-gsd.lbl.gov/vista) Vista genomic sequence alignment tool (Mayor et al. 2000) was used to compare the mouse and human Nanog genomic sequences. The promoter regions for mouse nanog and human Nanog and NanogGP1 were amplified from genomic DNA of the appropriate species. Primers for amplification, with restriction sites for cloning purposes indicated in lowercase, were:

nanog forward 5′-CGCgtcgacTAAAGTG AAATGAGGTAAAGCC-3′ (SEQ ID NO: 1),
nanog reverse 5′-CGCggatccGGAAAGATCATAGAA AGAAGAG-3′ (SEQ ID NO: 2),
Nanog forward 5′-CGGctcgagTTGCTCGGTTTTCTAGTTCC-3′(SEQ ID NO: 3),
Nanog reverse 5′-CGGctcgagCAAGAAATTGGGATAAAGTGAG-3′ (SEQ ID NO: 4),
NanogGP1 forward 5′-CGGctcgagTTGCTCGGTTTTCTAGTTCC-3′(SEQ ID NO: 5),
NanogGP1 reverse 5′-CGGctcgagCAAGAAGTTGTGATGAAGTGAG-3′ (SEQ ID NO: 6). The mouse nanog promoter fragment was cloned into both pGL3-Basic (Promega) and pEGFP1 (Clontech) vectors whereas the human promoters were cloned into pGL3-Basic alone. All constructs were sequence-verified.

Embryonic Stem Cell Culture and Reporter Lines

E14 mouse ESCs were grown in Dulbecco's modified Eagle's medium, 20% fetal bovine serum, 1× non-essential amino acids, 0.1 mM 2-mercaptoethanol, and an aliquot of recombinant LIF conditioned medium. ESCs were stably transfected with the NanogEGFP construct using a standard protocol, and individual colonies were picked after selection with 300 μg/ml G418 for 10 days with cells grown on neomycin-resistant mouse embryonic fibroblasts. Differentiation of ESCs was by withdrawal of LIF-conditioned medium, spontaneous differentiation into embryoid bodies, or the addition of retinoic acid. Fluorescence-activated cell sorting (FACS) was on a FACSCalibur (BD Biosciences). Quantitation of endogenous Nanog and enhanced green fluorescent protein (EGFP) reporter expression was by reverse transcription-PCR analyses in real time using the ABI PRISM 7900 sequence detection system (Applied Biosystems). Proligo-synthesized primer-probe sets for these were as follows:

Nanog forward, 5′-GGTTGAAGACTAGCAATGGTCTGA-3′ (SEQ ID NO: 7);
Nanog reverse, 5′-TGCAATGGATGCTGGGATACTC-3′ (SEQ ID NO: 8);
Nanog probe, 5′-TTCAGAAGGGCTCAGCACCA-3′ (SEQ ID NO: 9);
EGFP forward, 5′-CGACAACCACTACCTGAGCAC-3′ (SEQ ID NO: 10);
EGFP reverse, 5′-TCGTCCATGCCGAGAGTGAT-3′ (SEQ ID NO: 11);
EGFP probe, 5′-CGGCGGCGGTCACGAACTCCAGC-3′ (SEQ ID NO: 12). Expression was normalized to a-actin control (Applied Biosystems). The human ESC line HUES-6 (obtained from Doug Melton, Harvard University) was passaged according to Cowan et al. ([2004] N. Engl. J. Med. 350, 1353-1356).

### Example 1

### Analysis of the Effect of Downregulating Nanog

### (a) RNAi Analysis

The present example illustrates how the cellular effect of down-regulating the activity of the nanog gene may be analysed. The same method as in Example 6 (cf. below) showed that downregulation of Nanog leads to a differentiation of ESCs.

Gene-specific oligonucleotides for RNAi were designed according to Reynolds et al (2004) and Ui-Tei et al. (2004). The 19-nucleotide hairpin-type shRNAs with a 9-nucleotide loop were cloned into pSUPER.puro (Bgl II and Hind III sites, Oligoengine). The oligonucleotides used were as follows:

for GFP RNAi (control), 5′-GATCCCCGAACGGCATCAAGGTGAACTTCAAGAGAGTTCACCTTGATGCC GTTCTTTTTA-3′ (SEQ ID NO: 13) and 5′-AGCTTAAAAAGAACGGCATCAAGGTGAACTCTCTTGAAGTTCACCTTGAT GCCGTTCGGG-3′ (SEQ ID NO: 14);
for Nanog RNAi 5′-GATCCCCGAACTATTCTTGCTTACAATTCAAGAGATTGTAA GCAGAATAGTTCTTTTTA-3′ (SEQ ID NO: 15) and 5′-AGCTTAAAAAGAACTATTCTTGCTTACAATCTCTTGAATTGTAAGCAAGAA TAGTTCGGG-3′(SEQ ID NO: 16). E14 ES cells were co-transfected with shRNA plasmids and GFP plasmids at confluency of 50%.

### (b) Western Blot Analysis

In order to test whether Nanog RNAi specifically downregulated Nanog expression without affecting other transcription factors such as Sox2 or Oct4, protein levels were analysed by means of SDS-PAGE and subsequent Western Blot, two methods well known in the art. The Nanog expression vector was co-transfected with constructs expressing control, Nanog, Oct4 or Sox2 siRNA into 293T cells. Western blot analysis of cell lysates was performed with anti-Nanog or anti-β actin antibodies. β actin served as a loading control.

Proteins were separated on 12% SDS-PAGE gel and transferred to Immobilon P blotting membrane (Millipore) according to standard methods well established in the art. The membrane was blocked with PBS plus 0.1% tween 20 and 5% milk and probed with specific antibodies. Subsequently the membranes were incubated with horseradish-peroxidase-conjugated secondary antibodies. The blots were developed with ECL Advance Western Blotting Detection Kit (Amersham).

As FIG. 1A shows, only Nanog siRNA could reduce Nanog protein levels. Control RNAi, Oct4 RNAi and Sox2 RNAi (cf. Example 6) did not affect expression of Nanog. This Nanog siRNA did not affect the level of co-expressed Sox2 or Oct4 (cf. FIG. 1 B, C). These data show that the Nanog siRNA was specific towards Nanog, and did not affect two other transcription factors selected as model proteins for a POU- and homeo-domain-containing transcription factor and an HMG domain-containing transcription factor.

ES cells were transfected with the construct expressing Nanog or GFP siRNA (cf. also below). The transfected cells were selected with puromycin and Western blotting showed that the level of Nanog was largely reduced, i.e. to protein levels at the detection limit (FIG. 1D).

In addition, the typical morphology of undifferentiated ES cell was lost after Nanog knockdown (FIG. 1E). A conventional microscope was used for taking the respective photos. Alkaline phosphatase staining was also drastically reduced in Nanog knockdown cells.

### Example 2

### Verification of the Formation of a Complex Between NANOG and a Transcription Factor Containing a (POU)-Specific Domain and a Homeo-Domain and of a Transcription Factor Containing a HMG Domain In Vitro

To determine whether Oct4 and Sox2 were able to form a complex with an element in the nanog promoter, an assay known in the art as electrophoretic mobility shift assay (EMSA) was employed.

For this purpose nuclear extracts were prepared from E14 mouse embryonic stem cells grown on mouse embryonic fibroblast feeders using the method of Dignam et al. ([1983] Nucleic Acids Res. 11, 1475-1489) with modifications as described: cells were washed and harvested by scraping in PBS, then resuspended in 5 pellet volumes of buffer A (10 mM Hepes pH 7.9, 1.5 mM MgCl₂, 10 mM KCl, 0.5 mM DTT, 1% Protease Inhibitor Cocktail (Sigma)) and incubated on ice for 10 min. Cells were centrifuged and resuspended in 2 pellet volumes of buffer A then plasma membranes were lysed using 20 strokes of a Dounce homogenizer (type B pestle). Nuclei were collected by centrifugation and resuspended in 0.6 nuclear pellet volumes of buffer C (20 mM Hepes pH 7.9, 25% glycerol, 420 mM NaCl, 1.5 mM MgCl₂, 0.2 mM EDTA, 0.5 mM DTT, 1% Protease Inhibitor Cocktail) then incubated at 4° C. with rotation for 30 min. Nuclei were then centrifuged at 13000 rpm for 5 min and the supernatants were collected and dialyzed against dialysis buffer (20 mM Hepes pH 7.9, 20% glycerol, 100 mM KCl, 0.83 mM EDTA, 1.66 mM DTT, 1% Protease Inhibitor Cocktail) 4° C. for 2 h. Extracts were then stored at −80° C.

EMSA was performed using as a DNA probe a 37 bp double-stranded oligonucleotide containing a sequence from the nanog promoter overlapping the putative element designated NOS. Sox2 was selected as the transcription factor containing a (POU)-specific domain and a homeo-domain, and Oct4 was selected as a transcription factor containing a HMG domain. As a positive control the known Oct4/Sox2 composite binding site from the FGF4 enhancer (called FOS) was used, which, unlike the nanog element, has a spacing of 3 bp between the octamer and HMG motifs.

Double-stranded DNA oligonucleotides labeled with cy5 at the 5′ termini of both strands were used. Single-stranded, 5′-labeled oligonucleotides were purchased from Proligo and annealed to their complimentary 5′-labeled oligonucleotide by gradual cooling from 95° C. to room temperature. The sequences of the oligonucleotides used were as follows:

### FOS, 5′-TTTAAGTATCCCATTAGCATCCAAACAAAGAGTTTTC-3′ (SEQ ID NO: 17);

### NOS, 5′-CTTACAGCTTCTTTTGCATTACAATGTCCATGGTGGA-3′ (SEQ ID NO: 18),

### NmOS,5′-CTTACAGCTTCTTTCAAATTACAATGTCCATGGTGGA-3′ (SEQ ID NO: 19);

### NOmS, 5′-CTTACAGCTTCTTTTGCATTAACCTGTCCATGGTGGA-3′ (SEQ ID NO: 20);

### NmOmS, 5′-CTTACAGCTTCTTTCAAATTAACCTGTCCATGGTGGA-3′ (SEQ ID NO: 21);

NNS, 5′-CTGCAGGTGGGATTAACTGTGAATTCA-3′ (SEQ ID NO: 22) (cf. FIG. 4A). For DNA-binding reactions 2 μl (approximately 16 μg) of nuclear extract was added to a 10 μl reaction volume (final) containing 50 in M double-stranded labeled oligonucleotide and 5 μg poly-dGdC (Amersham). The final binding buffer composition was 60% dialysis buffer. Where specified 1 μM unlabeled oligonucleotide competitor was also included prior to addition of nuclear extracts. Binding reactions were carried out for 20 min at RT. Where specified 2 μl (anti-Oct4 and anti-JunB) or 8 μl (anti-Sox2 and anti-Sox4) of antibody was added following the initial binding reaction and incubated for an additional 20 min. Antibodies, purchased from Santa Cruz, were as follows: rabbit anti-Oct4 polyclonal (H-134) sc-9081x; goat anti-Sox2 polyclonal (Y-17) sc-17320; rabbit anti-JunB polyclonal (N-17) sc-46x; goat anti-Sox4 polyclonal (C-20) sc-17326. Binding reactions were separated on pre-run 6% native polyacrylamide gels in 0.5×TBE using a Bio-Rad protean II xi apparatus for 2 h at 300 V. Gels were imaged directly in glass plates using a Molecular Dynamics Typhoon 9140 phosphoimager using a red laser (633 nm) and cy5 emission filter with a PMT setting of 600 V, normal sensitivity and +3 mm focal plane. EMSA performed with mouse embryonic fibroblast feeders alone produced no significant mobility shifts, indicating that they did not contribute to the observed protein-DNA complexes.

Initially, it was tested whether DNA-binding proteins in nuclear extracts from E14 mouse embryonic stem cells could recognize the NOS element. As shown in FIG. 2, four major protein complexes were observed binding to the NOS element in EMSA. Four similarly migrating complexes were observed binding to the FOS element however one additional complex was also observed binding to this sequence (lane 12). Furthermore, complex C appeared to have a slightly faster mobility on the NOS element than on the FOS element.

To determine if any of these bands represented Oct4 or Sox2, it was tested whether adding antibodies for these proteins to the binding reactions resulted in supershifts. Indeed, following addition of an anti-Oct4 antibody two of the complexes, A and C, disappeared and labeled probe accumulated in the well (labeled Oct4 supershift). This was observed with both the NOS and FOS probes (lane 2 and 13) indicating that complexes A and C on both sequences contained Oct4. Similarly, addition of an anti-Sox2 antibody resulted in the disappearance of complex C with the NOS probe and both complexes C and E with the FOS probe (lanes 3 and 14). In both cases, a more slowly migrating complex (labeled Sox2 supershift) was observed. This indicates that complexes C and E contained Sox2. Importantly, addition of antibodies against either JunB or Sox4 had no effect on the mobility of the bands indicating the effects of the Oct4 and Sox2 antibodies were specific. Therefore, since complex C super-shifted with both the Oct4 and Sox2 antibodies this band represents the heterodimer of these proteins bound to the composite elements. As complex A migrated more quickly than the heterodimer and was recognized by only the Oct4 antibody, it strongly suggests that this complex is the Oct4 monomer. Likewise complex E on the FOS sequence migrated faster than the heterodimer and was recognized by only the Sox2 antibody indicating it represents a Sox2 monomer. The relative mobilities of these bands are consistent with previously published results identifying Oct4 and Sox2 in extracts of F9 teratocarcinoma cells binding to the FGF4 element.

At present, the identities of the factors binding in complexes B and D have not been established. A strong candidate for complex D is the transcription factor Oct1, which has been shown to recognize the octamer motif in the FGF4 element and to migrate with a similarly slow mobility. Moreover, Oct1 has also been shown to heterodimerize with Sox2 on the FGF4 composite element. However, the lack of an even more slowly migrating complex would suggest that this does not occur in E14 nuclear extracts, at least to levels detectable by EMSA.

The slightly faster mobility of the Oct4/Sox2 heterodimer binding to NOS compared to that binding FOS (compare complex C, lanes 1 and 12) is consistent with the idea that a more closed conformation could occur on the NOS sequence due to the absence of any nucleotides separating the octamer motif from the HMG motif, as occurs in the FOS sequence. It is tempting to speculate that such a differential conformation could result in a differential transcriptional activity or differential recruitment of co-regulatory factors.

Oligonucleotide competitions were performed in order to establish the DNA-binding specificity of these factors. Binding by all factors to NOS was strongly competed by addition of a 20-fold excess of the identical unlabeled NOS (lane 7) while a non-specific unlabeled oligonucleotide containing an immediately downstream sequence from the nanog promoter (NNS) did not effectively compete for binding (lane 11). These results indicate that binding by these factors to NOS is specific. Likewise binding to the FOS sequence is also specific as unlabeled FOS was able to compete all binding to the labeled FOS (lane 17) while the NSS was not (lane 22). Moreover, both unlabeled FOS and NOS are able to compete with each other (lane 6 and 18) indicating that competitors with either 0 or 3 bp spacing are equally effective when added at a 20-fold excess. With both NOS and FOS, if binding is performed in the presence of a competitor with a 3 bp substitution in the octamer motif, competition for binding by the Oct4 monomer and the Oct4/Sox2 heterodimer no longer occurs (lane 8 and 19) while competition for the Sox2 monomer still occurs (lane 19). This indicates that an intact octamer motif is required for binding of both the Oct4 monomer and the Oct4/Sox2 heterodimer. Likewise a 3 bp substitution in the HMG motif of a competitor results in the loss of competition for the Oct4/Sox2 heterodimer on NOS and FOS (lane 9 and 20) and the Sox2 monomer on FOS (lane 20). In both cases the Oct4 monomer is still competed. This indicates that an intact HMG domain is required for binding of the Sox2 monomer and the Sox2/Oct4 heterodimer. Finally, substitution of 3 bp in both the octamer and HMG motifs in the same competitor results in a loss of competition for the Oct4 monomer and Oct4/Sox2 heterodimer binding to both NOS and FOS (lane 10 and 21) as well as the Sox2 monomer binding to FOS (lane 21). As can be seen from FIG. 2, an EMSA is a convenient means of verifying the formation between transcription factors and Nanog. Nevertheless, other means such as for instance a GST pull-down assay or Biacore may be employed.

### Example 3

### Verification of the Formation of a Complex Between NANOG and a Transcription Factor Containing a (POU)-Specific Domain and a Homeo-Domain and of a Transcription Factor Containing a HMG Domain In Vivo

A method of analyzing interactions between Nanog and a transcription factor in vivo is known to the person skilled in the art as a “Chromatin immunoprecipitation assay” or “ChIP assay”.

The present example illustrates the use of this method with Oct4 and Sox2 antibodies and nuclear extracts from mouse and human ESCs and in mouse ESCs differentiated with retinoic acid.

ChIP assays with E14 mouse ESCs were carried out as described (Wells, J., and Farnham, P. J., (2002) Methods (Orlando) 26, 48-56). Briefly, cells were cross-linked with 1% formaldehyde for 10 min at room temperature, and formaldehyde was inactivated by the addition of 125 mM glycine. Chromatin extracts containing DNA fragments with an average size of 500 bp were immunoprecipitated using Oct4 (N19) or Sox2 (Y17) polyclonal antibodies (Santa Cruz Biotechnology) or a Sox2 (AB5603) polyclonal antibody (Chemicon). For all ChIP experiments, quantitative PCR analyses were performed in real time using the ABI PRISM 7900 sequence detection system (Applied Biosystems) and SYBR Green master mix (Applied Biosystems) as described (Ng, H. H., et al., (2003) Mol. Cell 11, 709-719). Relative occupancy values were calculated by determining the apparent immunoprecipitate efficiency (ratios of the amount of immunoprecipitated DNA over that of the input sample) and normalized to the level observed at a control region, which was defined as 1.0. Primer pairs were as follows:

1,

(SEQ ID NO: 23)

5′-GGCAAACTTTGAACTTGGGATGTGGAAATA-3′,

(SEQ ID NO: 24)

5′-CTCAGCCGTCTAAGCAATGGAAGAAGAAAT-3′;
2,

(SEQ ID NO: 25)

5′-GAGGATGCCCCCTAAGCTTTCCCTCCC-3′,

(SEQ ID NO: 26)

5′-CCTCCTACCCTACCCACCCCCTATTCTCCC-3′;
3,

(SEQ ID NO: 27)

5′-GGGTCACCTTACAGCTTCTTTTGCATTA-3′,

(SEQ ID NO: 28)

5′-GGCTCAAGGCGATAGATTTAAAGGGTAG-3′;
4,

(SEQ ID NO: 29)

5′-GGTGATACGTTGGCCTTCTAGTCTGAA-3′,

(SEQ ID NO: 30)

5′-GGGCAAATTGCAAACTAACTGTATAACCTC-3′.

From undifferentiated mouse ESCs grown in feeder-free conditions, DNA fragments containing the composite oct-sox element were enriched up to 43- and 37-fold when immunoprecipitated with the Oct4 and Sox2 antibodies, respectively (FIG. 3A, Amplicon 2). Two neighboring regions that did not contain the oct-sox composite element were not significantly enriched (FIG. 3A, Amplicons 1 and 3). Furthermore, upon retinoic acid-induced differentiation of mouse ESCs, this enrichment of the oct-sox-containing fragments was reduced corresponding to the degree of differentiation. After 3 days of differentiation, enrichment only reached a maximum of 10-fold above background with both Oct4 and Sox2 antibodies, and after 6 days of differentiation, no significant enrichment was detectable (FIG. 3A). Using an antibody to M11 as a negative control, there was no significant enrichment for any of the amplicons from any of the three ESC states analyzed (data not shown). The level of enrichment identified here corresponds well with the activity of the Nanog promoter in a similar differentiation protocol described above with the EGFP reporter line.

A similar ChIP analysis may be performed to verify that OCT4 and SOX2 also interact with the NANOG promoter in human ESCs. In the present example the human ESC line HUES-6 was used, grown on inactivated mouse embryonic fibroblasts. Six different amplicons were used (FIG. 3B), all within close proximity to exon 1 of NANOG, to detect for enrichment of DNA chromatin-immunoprecipitated with OCT4 and SOX2 antibodies. Only the three closest amplicons to the composite sox-oct element showed significant enrichment with both antibodies (FIG. 3B). A second Sox2 antibody (Y17) gave similar results (data not shown). The lower fold enrichment for all three antibodies (6-fold) as compared with that seen in mouse ESCs may be due to the presence of more differentiated cells within the human culture as undifferentiated human ESCs tend to be more difficult to maintain in culture. Using a glutathione S-transferase antibody as a negative control, there was no significant enrichment for any of the amplicons in these human ESCs (data not shown). In summary, all these ChIP data clearly indicate in vivo occupancy of Oct4/OCT4 and Sox2/SOX2 on the Nanog/NANOG promoter in undifferentiated mouse and human ESCs.

### Example 4

### Identification of a Binding Element on NANOG

The present example illustrates how a binding region on the Nanog gene can be identified based on its sequence conservation.

(a) Sequence Comparisons of a Non-Coding Region that Includes Cis-Regulatory Information

The present inventors had identified a highly conserved binding region of nanog, the sox-oct composite element. A pair-wise alignment of the mouse and human gene sequences had been generated utilizing the Vista on-line tool at LBL Berkley (Mayor, C. et al. (2000) Bioinformatics 16, 1046-1047), which takes into account species-specific repetitive elements in generating optimal alignments. For the alignment, the genomic region from 10 kb upstream of the most 5′ mouse nanog EST to 10 kb downstream of the most 3′ mouse nanog EST had been chosen. Peaks of sequence similarity were identified corresponding to the four exons of nanog, most significantly in the homeodomain-encoding exons 2 and 3 and least significantly in exon 1 (data not shown). Of the non-coding regions the only area of significant sequence conservation was found immediately upstream of the 5′-most EST in what was defined as the proximal promoter.

The lack of significant conservation in non-coding sequence elsewhere suggested the cis-regulatory elements directing pluripotent expression may all reside within this proximal promoter region. To test this, stably transfected mouse ES cell lines with a plasmid vector containing the mouse proximal promoter region driving the expression of a reporter EGFP were generated. The 406 bp promoter fragment consisted of sequence from −289 to +117 relative to what had been identified as the position of the furthest 5′ public EST which since has been defined as the predominant transcription start site (Hart, A. H. et al, supra). After selection the majority of these neo-resistant colonies fluoresced green indicating this region of the nanog promoter is indeed active in pluripotent cells (see FIG. 4B). A number of these nanogEGFP colonies were selected for further analysis.

In addition to the apparent pluripotent positive cis-regulatory elements within this promoter fragment the inventors were interested in determining whether it contained sufficient cis-regulatory information to down-regulate transcription upon ES cell differentiation as occurs with endogenous Nanog mRNA levels. Retinoic acid, a strong inducer of ES cell differentiation, was used at 0.1 μM to induce differentiation. This treatment drastically reduced EGFP expression after 3 days (cf. FIG. 4E-F) and by 4 days of differentiation only 9% of cells remained EGFP-positive as determined by FACS analysis (cf. FIG. 4G). This closely parallels endogenous nanog mRNA levels under the same differentiation protocol (Chambers, I. et al., supra).

Under subtler differentiation protocols the activity of this promoter construct continued to parallel that of endogenous Nanog mRNA levels. Culturing nanogEGFP cells in the absence of LIF reduced EGFP expression noticeably after 3 days of culture (see FIG. 4C-D), and by 4 days only 48% of cells were determined to be EGFP-positive compared to 74% from undifferentiated feeder-free nanogEGFP cells (cf. FIG. 4G). Embryoid body formation also induced the down-regulation of EGFP expression as measured by FACS analysis on a FACSCalibur (BD Biosciences). Quantitation of endogenous Nanog and EGFP reporter expression was performed by RT-PCR analyses as described above (cf embryonic stem cell culture and reporterlines). After the initial 2 days of differentiation EGFP expression progressively decreased and by 8 days represented only background levels of expression (cf. FIG. 4G), this is similar to the expression of endogenous Nanog mRNA (Hart et al. 2004). This 406 bp fragment of nanog thus contains sufficient cis-regulatory information to recapitulate endogenous nanog expression, at least as tested in an in vitro ES cell-based system.

### (b) Identification of a Binding Element by Phylogenetic Footprinting

Generating an alignment of the proximal promoter region from a diverse range of mammals enabled the identification a strong phylogenetic footprint. A very conservative estimate of the divergence time between the five species used in constructing this alignment, 12 million years ago for the mouse-rat split and 60 million years ago between each of the mouse, rat, human, cow, and elephant, provides a cumulative 252 million years of purifying selection to identify a footprint. The greatest stretch of sequence conservation was over a 94 bp region located from position −212 to −119 relative to the nanog transcription start site; within this 64 positions were invariant (cf. FIG. 5A).

A 16 bp stretch represented the longest uninterrupted invariant sequence and, intriguingly, within this was a 15 bp oct-sox composite element (cf. FIG. 5A) similar to those identified in and known to be functional for pluripotent expression of fgf4, utf1, sox2, and fbx15 (Yuan, H. et al., supra; Nishimoto, M. et al, supra; Tomioka, M. et al., supra; Tokuzawa, Y. et al., supra). The position of the sox and oct elements relative to each other is the same in all five of these genes which is indicative of the specific protein-protein contacts between the corresponding transcription factors (Sox2 and Oct4) known to bind these elements in pluripotent cells (Reményi, A. et al. (2003) Genes Dev. 17, 2048-2059). Similar to utf1, sox2, and fbx15, the sox and oct cis-elements in nanog are immediately adjacent to one another whereas in fgf4 3 bases separate the two elements.

Such strong purifying selection at numerous positions within the nanog promoter suggests a functional role for these positions in the regulation of transcription. Some of these positions, such as the oct-sox composite element, are likely to be important in pluripotent expression while others may represent cis-regulatory elements functional in the post-implantation expression of nanog. Furthermore, there is no apparent association between location and orientation of the sox-oct composite element and the expression level of the corresponding gene (Table 1) as measured by massively parallel signature sequencing in mouse ESCs (Wei, C. L., et al. (2005) Stem Cells 23, 166-185).

### Example 5

### Regulation of Nanog Gene Expression by a Complex of NANOG, a Transcription Factor Containing a (POU)-Specific Domain and a Homeo-Domain and a Transcription Factor Containing a HMG Domain

This example illustrates the analysis of the effect of the formation of a complex between NANOG and a transcription factor containing a (POU)-specific domain and a homeo-domain and of a transcription factor containing a HMG domain on the activation of pluripotent cell transcription.

To study the role of specific sequence elements within this conserved promoter, a series of luciferase reporter constructs was constructed and transfected into F9 teratocarcinoma cells.

A DNA fragment (−289 to +117) was amplified (Promega Pfu polymerase) from mouse genomic DNA (2B gDNA) by PCR using primers pNanogF and pNanogR. A SalI/BamHI digested fragment of this was cloned into the SalI/BamHI site of pEGFP-1 (BD Biosciences Clontech). Continuing with this complete construct, a SacI/BamHI fragment, which includes 22 bp from the pEGFP-1 multiple cloning site, was cloned into the SacI/BglII site of pGL3Basic (Promega). The resultant plasmid was verified by sequencing, named pGL3Basic pNanog, and used for subsequent mutagenesis experiments. Reporter plasmids were modified using the Transformer Site-directed mutagenesis kit (Clontech) to incorporate 3 bp mutations. The modified plasmids were verified by sequencing. F9 cells (ATCC) were cultured using Dulbecco's modified Eagle medium with high glucose (Gibco) containing 15% standard fetal bovine serum (Hyclone) and 1% pen/strep. They were maintained at 37° C. with 5% CO₂. DNA was transfected into F9 cells with Lipofectamine 2000 (Invitrogen) using the company's protocol for 24-well format cell culture. A renilla luciferase plasmid (pRL-TK from Promega) was co-transfected as an internal control. After 24 hours, the cells were lysed, and the luciferase activity of the lysate was measured with the Dual-Luciferase Reporter Assay System (Promega), using the Centro LB960 96-well luminometer (Berthold Technologies). At the minimum, transfections were done in duplicate and on two independent occasions. Reporter plasmids were modified using the Transformer site-directed mutagenesis kit (Clontech) to incorporate 3-bp mutations which were subsequently verified by sequencing.

The wild-type 406 bp promoter fragment used to generate the nanogEGFP ES cells had significant transcriptional activity, as expected, and was used as the positive control set arbitrarily to 100% activity in all comparisons (see FIGS. 5B, 2C). A shorter version (−230 to +63) of this wild-type promoter beginning just 5′ to the conserved sequence in FIG. 5A maintained approximately 130% of the original promoter activity. This suggests a potential negative regulatory element contained within sequence excluded from this shorter promoter but also indicates that pluripotent enhancer elements are contained within the −230 to +63 region.

To test the orientation dependence of this conserved module, identical constructs but with this region (spanning −289 to −94) in either forward or reverse orientation relative to the immediate proximal promoter (−93 to +117) were constructed. 79% of wild-type activity was maintained in the forward and 48% in the reverse construct. As significant transcriptional activity was maintained in this conserved region when reversed it suggests the enhancer activity is orientation independent though the slightly lower activity may indicate subtle orientation effects.

Another measure of the functional significance of the conserved region was to test the homologous region from a second species, i.e. with human nanog. This construct transfected into the mouse F9 cells maintained 60% of the mouse transcriptional activity (Hs.1, cf. FIG. 5B) indicating substantial functional conservation. The inventors also tested the homologous region from the tandem duplicated copy of human nanog, that of NanogGP1. Interestingly, this maintained significant transcriptional activity (Hs.2, cf. FIG. 5B) despite the very low level of this transcript in human pluripotent cells suggesting the inactivating mutation of NanogGP1 is not in a cis-regulatory element but likely that found generating a stop codon 8 amino acids into the coding sequence (Booth, H. A. and Holland, P. W, supra).

### Example 6

### Analysis of the Regulation of Nanog Activity RNAi Analysis

This example illustrates the analysis of the regulation of Nanog promoter activity by the formation of a complex between NANOG and a transcription factor containing a (POU)-specific domain and a homeo-domain and of a transcription factor containing a HMG domain. In the present example, the effect of Oct4 and Sox2 on the pluripotent activity of the Nanog promoter was analysed.

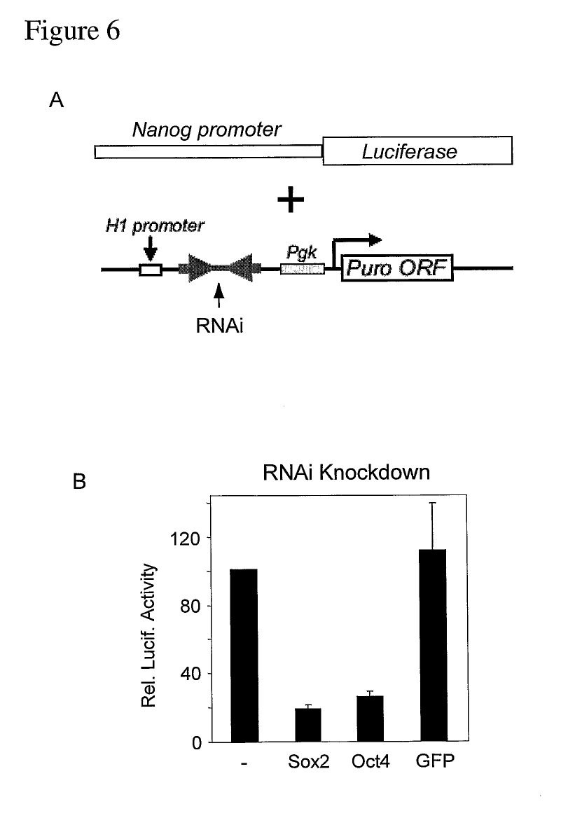

For this purpose, RNAi experiments were performed to reduce the level of Oct4 or Sox2. Both Pou5f1 and Sox2 RNAi constructs used in this study were previously shown to specifically knock down their respective mRNAs. A Nanog-luciferase reporter construct was co-transfected with Pou5f1, Sox2, GFP, or empty RNAi constructs (FIG. 6A) into mouse ESCs and subsequently assayed for luciferase activity.

Oligonucleotides were cloned into pSUPER.puro (BglII and HindIII sites; Oligoengine), which expresses 19-nucleotide hairpin-type short hairpin RNAs (shRNAs) with a 9-nucleotide loop, as described previously (Brummelkamp et al. 2002 Science 296, 550-553). All sequences were analyzed by BLAST search to ensure that they did not have significant sequence similarity with other genes. E14 mouse ESCs were seeded into 96-well plates at 20,000-30,000 cells/well density (with primary embryonic fibroblasts at 1000 cells/well) 1 day prior to transfection. Transfection was performed with Lipofectamine 2000. 50 ng of Nanog-pGL3 (firefly luciferase construct), 150 ng of pSuper or pSuper_RNAi, and phRLSV40 (normalization control expressing Renilla luciferase) were transfected.

Twenty-four hours after transfection, cells were selected for puromycin resistance for 2 further days prior to the luciferase activity being measured. The sequences used for RNAi were: Sox-2, 5′-GAAGGAGCACCCGGATTA-3′ (SEQ ID NO: 31); Oct4, 5′-GAAGGATGTGGTTCGAGT-3′ (SEQ ID NO: 32).

As can be seen in FIG. 6B, luciferase activity was reduced to almost that of background levels with the Pou5f1 and Sox2 RNAi, whereas the GFP and empty RNAi controls had no effect.

The inventions illustratively described herein may suitably be practiced in the absence of any element or elements, limitation or limitations, not specifically disclosed herein. Thus, for example, the terms “comprising”, “including”, “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by illustrative embodiments and optional features, modification and variation of the inventions embodied therein herein disclosed may be resorted to by those skilled in the art, and that such modifications and variations are considered to be within the scope of this invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims. In addition, where features or aspects of the invention are described in terms of Markush groups, those skilled in the art will recognize that the invention is also thereby described in terms of any individual member or subgroup of members of the Markush group.

TABLE 1
Molecular Features of Known Sox2-Oct4 Target Genes

| | | | | | | | MPSS |
|---|---|---|---|---|---|---|---|
| Gene^{a} | sox element | spacing | oct element | orientation | location^{b} | (bp) | level^{c}, tpm^{d} |
| Fgf4 | CTTTGTT | 3 | ATGCTAAT | forward | 3022 | 3′ | 101 |
| Utf1 | CATTGTT | 0 | ATGCTAGT | reverse | 1838 | 3′ | 2009 |
| Sox2 | CATTGTG | 0 | ATGCATAT | forward | 4041 | 3′ | 105 |
| Fbx15 | CATTGTT | 0 | ATGATAAA | reverse | 523 | 5′ | 129 |
| Nanog | CATTGTA | 0 | ATGCAAAA | reverse | 181 | 5′ | 112 |
| Pou5f1 | CTTTGTT | 0 | ATGCATCT | reverse | 1991 | 5′ | 388 |

^{a}references: Fgf4: Hart et al., supra; Utf1: Nishimoto et al., supra; Sox2: Tomioka et al., supra; Fbx15: Tokuzawa et al., supra; Nanog: the present inventors; Pou5f1: the present inventors, Okumura-Nakanishi, et al., supra
^{b}relative to the transcription start site
^{c}MPSS, massively parallel signature sequencing (Wei et al., supra)
^{d}tpm, tags per million in mouse ESCs

What is claimed is:

1. A method for maintaining pluripotency and/or self-renewing characteristics of stem/progenitor cells, the method comprising:
(a) contacting at least two transcription factors, or a functional fragment thereof, with the promoter region of the nanog gene, wherein one of the at least two transcription factors is selected from the POU- and homeo-domain-containing transcription factors, and wherein another of the at least two transcription factors is selected from the HMG domain-containing transcription factors; and
(b) allowing said at least two transcription factors to form a complex with a specific binding element within the nanog promoter, said complex regulating nanog gene expression by mediating transcriptional activation.

2. The method of claim 1, wherein the homeo-domain of said POU- and homeo-domain-containing transcription factor is a POU-homeo-domain.

3. The method of claim 1, or claim further comprising detecting said complex formation.

4. The method of claim 1, further comprising:
(c) measuring nanog gene expression in the stem/progenitor cells.

5. The method of claim 4, further comprising:
(d) comparing the result of the measurement obtained in step (c) with that of a control measurement.

6. The method of claim 1, wherein the at least two transcription factors form a heteromeric complex on the nanog promoter.

7. The method of claim 6, wherein the heteromeric complex is a heterodimer comprised of two transcription factors.

8. The method of claim 1, wherein the at least two transcription factors act synergistically to activate the transcription of the nanog gene.

9. The method of claim 1, wherein the POU- and homeo-domain-containing transcription factor is Oct4.

10. The method of claim 1, wherein the HMG-domain-containing transcription factor is Sox2.

11. The method of claim 1, wherein the at least two transcription factors bind to a composite binding element within the nanog promoter, with said POU- and homeo-domain-containing transcription factor binding to one part of the composite element, and said HMG domain-containing transcription factor binding to another part of the composite element.

12. The method of claim 11, wherein the composite binding element is a bipartite binding site.

13. The method of claim 12, wherein the composite binding element is an Oct4/Sox2 binding site.

14. The method of claim 13, wherein the Oct4 binding site and the Sox2 binding site are immediately adjacent to each other.

15. The method of claim 1, wherein the stem/progenitor cells are embryonic stem cells.

16. The method of claim 15, wherein the embryonic stem cells are of human origin.

17. The method of claim 1, further comprising contacting at least a third transcription factor with the nanog gene, wherein said third transcription factor is selected from the group consisting of a member of the SMAD protein family, a member of the AP1 family, a hand 1 transcription factor and a hand 1 related transcription factor.

18. The method of claim 1, wherein each of the at least two transcription factors bind to a binding site located within the region of the respective nanog gene that corresponds to the region of the human nanog gene that comprises sequence positions −289 to +117 of the human nanog gene.

19. The method of claim 18, wherein the binding site for each of the three transcription factors are located within the region of the nanog gene that corresponds to the region of the human nanog gene that comprises the sequence positions −212 to −119 of the human nanog gene.

20. A method for modulating gene expression in a cell, the method comprising:
(a) contacting at least two transcription factors or a functional fragment thereof with the promoter region of the nanog gene, wherein one of the at least two transcription factors is selected from the POU- and homeo-domain-containing transcription factors, and wherein another of the at least two transcription factors is selected from the HMG domain-containing transcription factors; and
(b) allowing said at least two transcription factors to form a complex with a specific binding element within the nanog promoter, said complex regulating gene expression by mediating transcriptional activation.

21. The method of claim 20, wherein the homeo-domain of said POU- and homeo-domain-containing transcription factor is a POU-homeo-domain.

22. The method of claim 20, wherein the cell is a stem/progenitor cell.

23. The method of claim 20, wherein the cell is an embryonic stem cell.

24. The method of claim 20, wherein the cell is a cancer cell.

25. The method of claim 24, wherein the cancer is a teratoma.

26. The method of claim 20, wherein the cell is a recombinant cell comprising a nanog promoter and expressing at least two transcription factors, wherein one of the at least two transcription factors is selected from the POU- and homeo-domain-containing transcription factors, and wherein another of the at least two transcription factors is selected from the HMG domain-containing transcription factors, or respectively a functional fragment thereof.

27. The method of claim 20, wherein the gene, the expression of which is modulated, is selected from the group consisting of a reporter gene, a drug resistance gene, an apoptosis gene (so-called ‘death’ gene), or and any other gene with desirable expression in a respective cell.

28. The method of claim 20, further comprising contacting said transcription factors with a compound that modulates the complex formation of said at least two transcription factors with said element of the nanog promoter.

29. The method of claim 28, wherein the complex formation of said at least two transcription factors or a functional fragment thereof with said element of the nanog promoter is reduced by means of a nucleic acid molecule.

30. The method of claim 29, wherein the nucleic acid molecule is RNA or DNA.

31. The method of claim 30, wherein the nucleic acid molecules is selected from the group consisting of an aptamer, a micro RNA (miRNA) molecule, a small interfering RNA (si-RNA) molecule or and a repeat-associated small interfering RNA (rasiRNA) molecule.

32. The method of claim 31, wherein the nucleic acid molecules is a si-RNA molecule transcribed from the sequence of SEQ ID NO: 31 and/or a si-RNA molecule transcribed from the sequence of SEQ ID NO: 32.

33. The method of claim 27, wherein the method is an in-vitro method for the identification of suitable compounds that modulate said complex formation.

34. The method of claim 33 for the in-vitro screening for potential compounds that are useful for removing or differentiating a cell due to their inhibition of the complex formation of said at least two transcription factors, or a functional fragment thereof, and said element of the nanog promoter, comprising the simultaneous screening of compound libraries on multiple-well microplates using automated work stations.

35. The method of claim 20, wherein said cell is comprised in a mammal and wherein the method is an in-vivo method.

36. The method of claim 35, wherein the mammal is selected from the group consisting of a rat, a mouse, a dingo, a cow, a goat, a pig, a chimpanzee, a macaque and a human.

37. The method of claim 35, comprising administering a compound (currently amended) that modulates the formation of said complex between the at least two transcription factors and the binding region of the nanog promoter.

38. The method of claim 35 for or in the treatment of teratoma.

39. The method of claim 20, further comprising detecting said complex formation.

40. The method of claim 20, further comprising (c) measuring nanog gene expression in the cell.

41. The method of claim 40, further comprising:
(d) comparing the result of the measurement obtained in step (c) with that of a control measurement.

42. The method of claim 3, wherein the detection is performed by a suitable spectroscopic, photochemical, photometric, fluorometric, radiological, enzymatic or thermodynamic method, or is based on cellular effects.

43. The method of claim 20, wherein the at least two transcription factors act synergistically to activate the transcription of the nanog gene.

44. The method of claim 20, wherein the POU- and homeo-domain-containing transcription factor is Oct4.


SEQUENCE LISTING (41 sequences)

SEQ ID NO: 1 - DNA, 31 bp, Artificial Sequence
  Description of Artificial Sequence Synthetic primer
cgcgtcgactaaagtgaaatgaggtaaagcc

SEQ ID NO: 2 - DNA, 31 bp, Artificial Sequence
  Description of Artificial Sequence Synthetic primer
cgcggatccggaaagatcatagaaagaagag

SEQ ID NO: 3 - DNA, 29 bp, Artificial Sequence
  Description of Artificial Sequence Synthetic primer
cggctcgagttgctcggttttctagttcc

SEQ ID NO: 4 - DNA, 31 bp, Artificial Sequence
  Description of Artificial Sequence Synthetic primer
cggctcgagcaagaaattgggataaagtgag

SEQ ID NO: 5 - DNA, 29 bp, Artificial Sequence
  Description of Artificial Sequence Synthetic primer
cggctcgagttgctcggttttctagttcc

SEQ ID NO: 6 - DNA, 31 bp, Artificial Sequence
  Description of Artificial Sequence Synthetic primer
cggctcgagcaagaagttgtgatgaagtgag

SEQ ID NO: 7 - DNA, 24 bp, Artificial Sequence
  Description of Artificial Sequence Synthetic primer
ggttgaagactagcaatggtctga

SEQ ID NO: 8 - DNA, 22 bp, Artificial Sequence
  Description of Artificial Sequence Synthetic primer
tgcaatggatgctgggatactc

SEQ ID NO: 9 - DNA, 20 bp, Artificial Sequence
  Description of Artificial Sequence Synthetic probe
ttcagaagggctcagcacca

SEQ ID NO: 10 - DNA, 21 bp, Artificial Sequence
  Description of Artificial Sequence Synthetic primer
cgacaaccactacctgagcac

SEQ ID NO: 11 - DNA, 20 bp, Artificial Sequence
  Description of Artificial Sequence Synthetic primer
tcgtccatgccgagagtgat

SEQ ID NO: 12 - DNA, 23 bp, Artificial Sequence
  Description of Artificial Sequence Synthetic probe
cggcggcggtcacgaactccagc

SEQ ID NO: 13 - DNA, 60 bp, Artificial Sequence
  Description of Artificial Sequence Synthetic oligonucleotide
gatccccgaacggcatcaaggtgaacttcaagagagttcaccttgatgccgttcttttta

SEQ ID NO: 14 - DNA, 60 bp, Artificial Sequence
  Description of Artificial Sequence Synthetic oligonucleotide
agcttaaaaagaacggcatcaaggtgaactctcttgaagttcaccttgatgccgttcggg

SEQ ID NO: 15 - DNA, 59 bp, Artificial Sequence
  Description of Artificial Sequence Synthetic oligonucleotide
gatccccgaactattcttgcttacaattcaagagattgtaagcagaatagttcttttta

SEQ ID NO: 16 - DNA, 60 bp, Artificial Sequence
  Description of Artificial Sequence Synthetic oligonucleotide
agcttaaaaagaactattcttgcttacaatctcttgaattgtaagcaagaatagttcggg

SEQ ID NO: 17 - DNA, 37 bp, Artificial Sequence
  Description of Artificial Sequence Synthetic probe
tttaagtatcccattagcatccaaacaaagagttttc

SEQ ID NO: 18 - DNA, 37 bp, Artificial Sequence
  Description of Artificial Sequence Synthetic probe
cttacagcttcttttgcattacaatgtccatggtgga

SEQ ID NO: 19 - DNA, 37 bp, Artificial Sequence
  Description of Artificial Sequence Synthetic probe
cttacagcttctttcaaattacaatgtccatggtgga

SEQ ID NO: 20 - DNA, 37 bp, Artificial Sequence
  Description of Artificial Sequence Synthetic probe
cttacagcttcttttgcattaacctgtccatggtgga

SEQ ID NO: 21 - DNA, 37 bp, Artificial Sequence
  Description of Artificial Sequence Synthetic probe
cttacagcttctttcaaattaacctgtccatggtgga

SEQ ID NO: 22 - DNA, 27 bp, Artificial Sequence
  Description of Artificial Sequence Synthetic probe
ctgcaggtgggattaactgtgaattca

SEQ ID NO: 23 - DNA, 30 bp, Artificial Sequence
  Description of Artificial Sequence Synthetic primer
ggcaaactttgaacttgggatgtggaaata

SEQ ID NO: 24 - DNA, 30 bp, Artificial Sequence
  Description of Artificial Sequence Synthetic primer
ctcagccgtctaagcaatggaagaagaaat

SEQ ID NO: 25 - DNA, 27 bp, Artificial Sequence
  Description of Artificial Sequence Synthetic primer
gaggatgccccctaagctttccctccc

SEQ ID NO: 26 - DNA, 30 bp, Artificial Sequence
  Description of Artificial Sequence Synthetic primer
cctcctaccctacccaccccctattctccc

SEQ ID NO: 27 - DNA, 28 bp, Artificial Sequence
  Description of Artificial Sequence Synthetic primer
gggtcaccttacagcttcttttgcatta

SEQ ID NO: 28 - DNA, 28 bp, Artificial Sequence
  Description of Artificial Sequence Synthetic primer
ggctcaaggcgatagatttaaagggtag

SEQ ID NO: 29 - DNA, 27 bp, Artificial Sequence
  Description of Artificial Sequence Synthetic primer
ggtgatacgttggccttctagtctgaa

SEQ ID NO: 30 - DNA, 30 bp, Artificial Sequence
  Description of Artificial Sequence Synthetic primer
gggcaaattgcaaactaactgtataacctc

SEQ ID NO: 31 - DNA, 18 bp, Artificial Sequence
  Description of Artificial Sequence Synthetic oligonucleotide
gaaggagcacccggatta

SEQ ID NO: 32 - DNA, 18 bp, Artificial Sequence
  Description of Artificial Sequence Synthetic oligonucleotide
gaaggatgtggttcgagt

SEQ ID NO: 33 - DNA, 95 bp, Mus musculus
ctccctcccagtctgggtcaccttacagcttcttttgcattacaatgtccatggtggaccctgcaggtgggattaactgtgaattcacagggctg

SEQ ID NO: 34 - DNA, 95 bp, Rattus sp.
ctccctctcagtctgggtcactttacagcttcttttgcattacaatgtccttggtgggcctggcaggagggattaactgtgaattcgcagggctg

SEQ ID NO: 35 - DNA, 95 bp, Homo sapiens
tccccacctagtctgggttactctgcagctacttttgcattacaatggccttggtgagactggtagacgggattaactgagaattcacaagggtg

SEQ ID NO: 36 - DNA, 96 bp, Bos sp.
tccccaccgggtctgggttactctgcaactctcttttgcattacaatggccttggtgagactggcagacgggattaactgggaattcgcaagggtg

SEQ ID NO: 37 - DNA, 107 bp, Elephantidae gen. sp.
tctccactcagtctgggttaccctgcagctctactttcaggaacttttgcattacaatggacttgatgagactggcagacaggattaactgggaattcaccagggtg

SEQ ID NO: 38 - protein, 10 aa, Artificial Sequence
  Description of Artificial Sequence Synthetic peptide
ASMTGGQQMG

SEQ ID NO: 39 - protein, 11 aa, Artificial Sequence
  Description of Artificial Sequence Synthetic peptide
QPELAPEDPED

SEQ ID NO: 40 - protein, 9 aa, Artificial Sequence
  Description of Artificial Sequence Synthetic peptide
YPYDVPDYA

SEQ ID NO: 41 - protein, 10 aa, Artificial Sequence
  Description of Artificial Sequence Synthetic peptide
EQKLISEEDL